SEQUENCE LISTING (10 sequences)

SEQ ID NO: 1 - protein, 1057 aa, Homo sapiens
QLSYGYDEKSTGGISVPGPMGPSGPRGLPGPPGAPGPQGFQGPPGEPGEPGASGPMGPRGPPGPPGKNGDDGEAGKPGRPGERGPPGPQGARGLPGTAGLPGMKGHRGFSGLDGAKGDAGPAGPKGEPGSPGENGAPGQMGPRGLPGERGRPGAPGPAGARGNDGATGAAGPPGPTGPAGPPGFPGAVGAKGEAGPQGPRGSEGPQGVRGEPGPPGPAGAAGPAGNPGADGQPGAKGANGAPGIAGAPGFPGARGPSGPQGPGGPPGPKGNSGEPGAPGSKGDTGAKGEPGPVGVQGPPGPAGEEGKRGARGEPGPTGLPGPPGERGGPGSRGFPGADGVAGPKGPAGERGSPGPAGPKGSPGEAGRPGEAGLPGAKGLTGSPGSPGPDGKTGPPGPAGQDGRPGPPGPPGARGQAGVMGFPGPKGAAGEPGKAGERGVPGPPGAVGPAGKDGEAGAQGPPGPAGPAGERGEQGPAGSPGFQGLPGPAGPPGEAGKPGEQGVPGDLGAPGPSGARGERGFPGERGVQGPPGPAGPRGANGAPGNDGAKGDAGAPGAPGSQGAPGLQGMPGERGAAGLPGPKGDRGDAGPKGADGSPGKDGVRGLTGPIGPPGPAGAPGDKGESGPSGPAGPTGARGAPGDRGEPGPPGPAGFAGPPGADGQPGAKGEPGDAGAKGDAGPPGPAGPAGPPGPIGNVGAPGAKGARGSAGPPGATGFPGAAGRVGPPGPSGNAGPPGPPGPAGKEGGKGPRGETGPAGRPGEVGPPGPPGPAGEKGSPGADGPAGAPGTPGPQGIAGQRGVVGLPGQRGERGFPGLPGPSGEPGKQGPSGASGERGPPGPMGPPGLAGPPGESGREGAPGAEGSPGRDGSPGAKGDRGETGPAGPPGAPGAPGAPGPVGPAGKSGDRGETGPAGPAGPVGPVGARGPAGPQGPRGDKGETGEQGDRGIKGHRGFSGLQGPPGPPGSPGEQGPSGASGPAGPRGPPGSAGAPGKDGLNGLPGPIGPPGPRGRTGDAGPVGPPGPPGPPGPPGPPSAGFDFSFLPQPPQEKAHDGGRYYRA

SEQ ID NO: 2 - protein, 1057 aa, synthetic construct
  source 1..1057: note = Amino acid sequence of a1 (I) M1
QLSYGYDEKSTGGISVPGPMGPSGPRGLPGPPGAPGPQGFQGPPGEPGEPGASGPPGPPGPPGPPGKNGDDGEAGKPGRPGERGPPGPQGARGLPGTAGLPGMKGHRGFSGLDGAKGDAGPAGPKGEPGSPGENGAPGQPGPPGLPGERGRPGAPGPAGARGNDGATGAAGPPGPTGPAGPPGFPGAVGAKGEAGPQGPRGSEGPQGVRGEPGPPGPAGAAGPAGNPGADGQPGAKGANGAPGIAGAPGFPGARGPSGPQGPGGPPGPKGNSGEPGAPGSKGDTGAKGEPGPVGVQGPPGPAGEEGKRGARGEPGPTGLPGPPGERGGPGSRGFPGADGVAGPKGPAGERGSPGPAGPKGSPGEAGRPGEAGLPGAKGLTGSPGSPGPDGKTGPPGPAGQDGRPGPPGPPGARGQAGVMGFPGPKGAAGEPGKAGERGVPGPPGAVGPAGKDGEAGAQGPPGPAGPAGERGEQGPAGSPGFQGLPGPAGPPGEAGKPGEQGVPGDLGAPGPSGARGERGFPGERGVQGPPGPAGPRGANGAPGNDGAKGDAGAPGAPGSQGAPGLQGMPGERGAAGLPGPKGDRGDAGPKGADGSPGKDGVRGLTGPIGPPGPAGAPGDKGESGPSGPAGPTGARGAPGDRGEPGPPGPAGFAGPPGADGQPGAKGEPGDAGAKGDAGPPGPAGPAGPPGPIGNVGAPGAKGARGSAGPPGATGFPGAAGRVGPPGPSGNAGPPGPPGPAGKEGGKGPRGETGPAGRPGEVGPPGPPGPAGEKGSPGADGPAGAPGTPGPQGIAGQRGVVGLPGQRGERGFPGLPGPSGEPGKQGPSGASGERGPPGPMGPPGLAGPPGESGREGAPGAEGSPGRDGSPGAKGDRGETGPAGPPGAPGAPGAPGPVGPAGKSGDRGETGPAGPAGPVGPVGARGPAGPQGPRGDKGETGEQGDRGIKGHRGFSGLQGPPGPPGSPGEQGPSGASGPAGPRGPPGSAGAPGKDGLNGLPGPIGPPGPRGRTGDAGPVGPPGPPGPPGPPGPPSAGFDFSFLPQPPQEKAHDGGRYYRA

SEQ ID NO: 3 - DNA, 3171 bp, synthetic construct
  source 1..3171: mol_type = other DNA; note = Nucleotide sequence encoding a1 (I) M1
caacttagttatggatacgatgaaaaatccacaggtggaatcagtgttcctggacctatgggtccatcaggtccaagaggtttaccaggacctccaggtgccccaggtccccagggatttcaaggtccaccaggagagcctggtgagccaggagcttctggtccacctggtccccctggaccacctggtcctccaggaaagaatggagatgatggtgaagctggaaaacctggaagacctggagaaagaggaccaccaggaccccagggtgccagaggactgccaggtaccgcaggtctgcctggaatgaaaggtcatagaggattttcaggattagacggtgcaaagggagacgctggacctgcaggaccaaagggtgagccaggaagtccaggagagaatggtgcaccaggacagccaggtccacctggactgcccggtgaaagaggtagacccggagcaccaggaccagcaggtgcaagaggaaatgatggagctacaggtgctgcaggacccccaggtccaacaggaccagccggtcctcccggtttcccaggtgccgttggagcaaaaggtgaagctggtccacagggtccaagaggttctgaaggtccacagggagttagaggagaaccaggaccccctggaccagctggtgcagcaggaccagctggtaaccctggtgctgacggtcagccaggtgctaagggagcaaatggagcaccaggaatagctggtgccccaggatttcccggtgctagaggtccaagtggtccacaaggaccaggaggtccacccggtcccaaaggaaacagtggagaaccaggtgcacccggttcaaagggagatacaggagctaaaggagagcccggtccagtgggtgttcagggaccacccggacctgctggagaggaaggtaaaagaggtgcaagaggtgagccaggaccaacaggtctgcctggtccccctggtgaaagaggtggtccaggtagtagaggatttccaggagctgatggtgttgcaggaccaaagggacccgcaggtgagagaggatcacccggtccagccggaccaaaaggatcaccaggagaagctggtagaccaggagaagctggtctgccaggtgctaaaggattgacaggatcacccggttcacctggtcctgatggaaagacaggacctccaggtcccgctggtcaggacggtagaccaggacccccaggacccccaggtgcaagaggtcaggcaggtgtaatgggtttccccggacctaaaggagcagctggagaacctggtaaagctggagagagaggagtgcctggaccccctggagctgttggtccagcaggaaaggatggtgaggcaggtgcacaaggtccacctggacccgctggacctgcaggtgagagaggagagcaaggtcccgcaggttctccaggttttcagggtttgccaggtccagccggtcctcctggagaggcaggaaagccaggagaacaaggagttccaggagacctgggtgcaccaggaccctctggtgcaagaggagagagaggatttcctggagaaagaggtgtgcagggaccaccaggtcccgccggtccaagaggagcaaatggagcccctggaaatgacggagctaagggtgacgctggtgcaccaggagcaccaggttctcaaggtgctcccggattgcagggtatgcctggagagagaggtgcagctggactgccaggtccaaaaggtgacagaggagacgccggtcctaagggagctgacggttctcctggaaaggacggtgtgagaggtttgacaggaccaataggtccacccggtcctgctggagcccctggagacaaaggtgaatcaggtccttccggtccagccggaccaacaggagcaagaggagcacctggagacagaggagagccaggtcctccaggacctgcaggtttcgctggtcctcccggagcagatggacagccaggagctaagggagaacccggtgacgctggtgctaagggagatgcaggtccaccaggtcctgctggtcctgctggacctcccggaccaataggtaatgttggagcacccggagcaaaaggtgccagaggttccgcaggtcctcccggagcaactggttttccaggagctgccggaagagtgggtccacctggtccttctggaaatgcaggaccaccaggtcctcctggtccagccggaaaggaaggtggaaagggacctagaggagaaacaggtcccgcaggtagacccggtgaggtgggtccacctggtccacccggtccagctggtgagaaaggaagtcctggagcagacggaccagctggtgcccctggtacaccaggaccccaaggaatagctggtcaaagaggtgttgttggtttaccaggtcagagaggagaaagaggttttccaggattaccaggtccctcaggtgagcccggaaaacagggtccctcaggagcaagtggtgaaagaggaccaccaggaccaatgggacctccaggattagctggtccaccaggagaatcaggaagagagggtgctcctggagcagaaggttcaccaggaagagacggttcacccggagccaagggagacagaggtgaaacaggtcccgcaggtccaccaggagcacccggagcccctggtgctccaggacctgtcggaccagcaggaaaatccggtgacagaggtgagactggacccgcaggtcctgctggtcctgttggaccagtgggtgcaagaggaccagcaggtccacaaggtccaagaggtgacaaaggtgagacaggtgagcagggtgacagaggaattaaaggtcacagaggattttcaggactgcagggaccacccggtcctcccggttccccaggagagcaaggtccatccggtgcatccggtccagctggacccagaggaccacctggttctgctggtgcaccaggtaaagatggattgaacggtttgcctggtccaataggacctcctggtccaagaggaagaactggtgacgccggtcccgtcggaccacccggtccaccaggtcccccaggtccacccggaccaccatccgcaggatttgatttctcattccttcctcaacctcctcaagagaaagcacatgatggaggtagatactatagagcc

SEQ ID NO: 4 - protein, 1060 aa, Homo sapiens
QMAGGFDEKAGGAQLGVMQGPMGPMGPRGPPGPAGAPGPQGFQGNPGEPGEPGVSGPMGPRGPPGPPGKPGDDGEAGKPGKAGERGPPGPQGARGFPGTPGLPGVKGHRGYPGLDGAKGEAGAPGVKGESGSPGENGSPGPMGPRGLPGERGRTGPAGAAGARGNDGQPGPAGPPGPVGPAGGPGFPGAPGAKGEAGPTGARGPEGAQGPRGEPGTPGSPGPAGASGNPGTDGIPGAKGSAGAPGIAGAPGFPGPRGPPGPQGATGPLGPKGQTGEPGIAGFKGEQGPKGEPGPAGPQGAPGPAGEEGKRGARGEPGGVGPIGPPGERGAPGNRGFPGQDGLAGPKGAPGERGPSGLAGPKGANGDPGRPGEPGLPGARGLTGRPGDAGPQGKVGPSGAPGEDGRPGPPGPQGARGQPGVMGFPGPKGANGEPGKAGEKGLPGAPGLRGLPGKDGETGAAGPPGPAGPAGERGEQGAPGPSGFQGLPGPPGPPGEGGKPGDQGVPGEAGAPGLVGPRGERGFPGERGSPGAQGLQGPRGLPGTPGTDGPKGASGPAGPPGAQGPPGLQGMPGERGAAGIAGPKGDRGDVGEKGPEGAPGKDGGRGLTGPIGPPGPAGANGEKGEVGPPGPAGSAGARGAPGERGETGPPGPAGFAGPPGADGQPGAKGEQGEAGQKGDAGAPGPQGPSGAPGPQGPTGVTGPKGARGAQGPPGATGFPGAAGRVGPPGSNGNPGPPGPPGPSGKDGPKGARGDSGPPGRAGEPGLQGPAGPPGEKGEPGDDGPSGAEGPPGPQGLAGQRGIVGLPGQRGERGFPGLPGPSGEPGKQGAPGASGDRGPPGPVGPPGLTGPAGEPGREGSPGADGPPGRDGAAGVKGDRGETGAVGAPGAPGPPGSPGPAGPTGKQGDRGEAGAQGPMGPSGPAGARGIQGPQGPRGDKGEAGEPGERGLKGHRGFTGLQGLPGPPGPSGDQGASGPAGPSGPRGPPGPVGPSGKDGANGIPGPIGPPGPRGRSGETGPAGPPGNPGPPGPPGPPGPGIDMSAFAGLGPREKGPDPLQYMRA

SEQ ID NO: 5 - protein, 1060 aa, synthetic construct
  source 1..1060: note = Amino acid sequence of a1 (II) M6
QMAGGFDEKAGGAQLGPPQGPPGPPGPPGPPGPAGAPGPQGFQGNPGEPGEPGVSGPPGPPGPPGPPGKPGDDGEAGKPGKAGERGPPGPQGARGFPGTPGLPGVKGHRGYPGLDGAKGEAGAPGVKGESGSPGENGSPGPPGPPGLPGERGRTGPAGAAGARGNDGQPGPAGPPGPVGPAGGPGFPGAPGAKGEAGPTGARGPEGAQGPRGEPGTPGSPGPAGASGNPGTDGIPGAKGSAGAPGIAGAPGFPGPRGPPGPQGATGPLGPKGQTGEPGIAGFKGEQGPKGEPGPAGPQGAPGPAGEEGKRGARGEPGGVGPIGPPGERGAPGNRGFPGQDGLAGPKGAPGERGPSGLAGPKGANGDPGRPGEPGLPGARGLTGRPGDAGPQGKVGPSGAPGEDGRPGPPGPQGARGQPGVMGFPGPKGANGEPGKAGEKGLPGAPGLRGLPGKDGETGAAGPPGPAGPAGERGEQGAPGPSGFQGLPGPPGPPGEGGKPGDQGVPGEAGAPGLVGPRGERGFPGERGSPGAQGLQGPRGLPGTPGTDGPKGASGPAGPPGAQGPPGLQGMPGERGAAGIAGPKGDRGDVGEKGPEGAPGKDGGRGLTGPIGPPGPAGANGEKGEVGPPGPAGSAGARGAPGERGETGPPGPAGFAGPPGADGQPGAKGEQGEAGQKGDAGAPGPQGPSGAPGPQGPTGVTGPKGARGAQGPPGATGFPGAAGRVGPPGSNGNPGPPGPPGPSGKDGPKGARGDSGPPGRAGEPGLQGPAGPPGEKGEPGDDGPSGAEGPPGPQGLAGQRGIVGLPGQRGERGFPGLPGPSGEPGKQGAPGASGDRGPPGPVGPPGLTGPAGEPGREGSPGADGPPGRDGAAGVKGDRGETGAVGAPGAPGPPGSPGPAGPTGKQGDRGEAGAQGPMGPSGPAGARGIQGPQGPRGDKGEAGEPGERGLKGHRGFTGLQGLPGPPGPSGDQGASGPAGPSGPRGPPGPVGPSGKDGANGIPGPIGPPGPRGRSGETGPAGPPGNPGPPGPPGPPGPGIDMSAFAGLGPREKGPDPLQYMRA

SEQ ID NO: 6 - DNA, 3180 bp, synthetic construct
  source 1..3180: mol_type = other DNA; note = Nucleotide sequence encoding a1 (II) M6
caaatggctggtggattcgatgaaaaggctggtggagcccaattaggtcctccacaaggtcctcccggtccacctggtcctcccggtcctccaggtcccgccggtgctcctggaccacagggtttccaaggaaaccccggtgaaccaggtgagcctggtgtttcaggtcctcccggtcctccaggaccacctggaccaccaggaaagcctggtgacgacggagaagctggtaaaccaggaaaggcaggagagagaggtccacctggacctcagggtgccagaggtttcccaggtacccctggtcttcctggtgtcaagggtcatagaggttaccccggtttggatggtgccaagggtgaagccggtgcccctggtgttaagggtgaatcaggaagtcccggtgaaaatggaagtcccggtccacccggtccacctggactgccaggtgagagaggaagaaccggaccagctggtgctgcaggtgctagaggaaatgacggacagcccggaccagccggacctcccggtcctgttgggcccgcaggtggtcctggtttccctggtgctcctggagccaaaggagaagccggacccaccggagccagaggtcccgagggagcacagggacctagaggagaaccaggtacaccaggtagtcccggtcctgctggtgcatcaggaaatcccggaactgacggtattccaggagcaaagggatctgcaggagcaccaggaatagctggtgctcctggatttccaggtcccagaggacctcccggtcctcaaggagcaacaggtcctttgggaccaaaaggtcaaacaggagaaccaggtattgctggattcaaaggagagcaaggtccaaagggagagcccggtcccgcaggtccccaaggagccccaggaccagctggtgaagaaggaaaaagaggagccagaggtgaacctggaggagtaggacctattggtcctcctggtgagagaggtgctcccggaaacagaggttttcctggtcaagatggtctggctggacctaaaggtgctccaggagagagaggaccttcaggacttgctggtccaaaaggtgctaacggagatccaggaagacccggtgaacctggtctgcctggagctagaggattaacaggaagaccaggtgacgcaggtccccagggtaaagtgggtcccagtggtgccccaggtgaagatggaagacctggtcctcccggaccccaaggtgcaagaggtcagcctggagtgatgggatttcctggacccaagggtgctaacggagaacctggaaaagctggtgagaaaggactgcccggtgccccaggtcttagaggtttgccaggtaaagatggagaaacaggagccgcaggaccacccggtccagccggaccagcaggagagagaggtgaacaaggagcacctggtccaagtggttttcagggtcttccaggtccccctggtccaccaggagagggaggtaaaccaggtgaccaaggtgtccctggagaagcaggtgcacccggtcttgtgggtccaagaggtgaaagaggattccctggtgagagaggatctcccggagcccagggacttcaaggtcctagaggtctgccaggtacccctggtacagacggaccaaagggagcatcaggacccgctggacctcccggagcccaaggtcctccaggtttacaaggtatgcctggtgaaagaggtgctgcaggtatagctggaccaaaaggagacagaggtgacgttggtgagaagggtcccgaaggagcccctggaaaagatggtggaagaggattaacaggtcctataggaccacccggtccagccggtgctaatggagaaaaaggagaagtaggtcctccaggtccagcaggatctgcaggtgctagaggtgcccctggagagagaggtgaaacaggaccacctggtccagctggtttcgctggtcccccaggagctgatggacagcccggtgcaaaaggtgaacaaggagaagccggacagaagggagatgctggagcccccggtccacaaggtccctcaggagcaccaggtcctcaaggtccaactggtgtgaccgggccaaagggtgcaagaggagcacagggacctccaggagcaacaggtttcccaggagctgctggtagagtcggtccacccggatctaatggtaaccccggaccaccaggaccacctggaccatctggaaaggatggacccaaaggagcaagaggagattcaggaccacccggaagagcaggagaacctggattacagggtcccgccggtccaccaggagagaaaggagagcccggagatgatggtccctcaggtgcagagggacccccaggaccccaaggtctggcaggtcaaagaggtatagtgggtcttccaggtcaaagaggtgaaagaggatttccaggacttccaggtccttcaggtgaacccggtaaacagggagcccccggagcctcaggtgacagaggtcctccaggaccagtaggacccccaggtttaaccggaccagcaggtgagccaggaagagaaggttctcctggagccgatggacctccaggaagagacggtgcagctggtgttaagggtgacagaggtgaaactggagccgtaggagccccaggtgcccccggaccacccggatcacccggacctgcaggtcctactggtaaacaaggagatagaggagaagccggtgcccagggtcctatgggtccttctggtcctgcaggagcaagaggtatacaaggtccacagggtcccagaggtgacaagggtgaagcaggagaacccggtgagagaggtctgaagggtcatagaggattcaccgggttacagggtttgccaggaccccctggaccaagtggtgaccagggtgcatccggtccagcaggtccttctggaccaagaggtcctcccggtccagttggtccatcaggtaaagacggagccaacggtatcccaggtcccatcggtcctccaggtcctagaggaagaagtggagagactggtcctgctggacctcctggaaaccctggtcctccaggacctccaggtcctccaggtcccggaatagatatgtccgctttcgctggattgggaccaagagagaaaggtcctgaccctcttcaatatatgagagca

SEQ ID NO: 7 - protein, 1071 aa, synthetic construct
  source 1..1071: note = Amino acid sequence of a1 (I) M1 with N-terminal Strep-Tag II tag and C-terminal 6 x His Tag
WSHPQFEKQLSYGYDEKSTGGISVPGPMGPSGPRGLPGPPGAPGPQGFQGPPGEPGEPGASGPPGPPGPPGPPGKNGDDGEAGKPGRPGERGPPGPQGARGLPGTAGLPGMKGHRGFSGLDGAKGDAGPAGPKGEPGSPGENGAPGQPGPPGLPGERGRPGAPGPAGARGNDGATGAAGPPGPTGPAGPPGFPGAVGAKGEAGPQGPRGSEGPQGVRGEPGPPGPAGAAGPAGNPGADGQPGAKGANGAPGIAGAPGFPGARGPSGPQGPGGPPGPKGNSGEPGAPGSKGDTGAKGEPGPVGVQGPPGPAGEEGKRGARGEPGPTGLPGPPGERGGPGSRGFPGADGVAGPKGPAGERGSPGPAGPKGSPGEAGRPGEAGLPGAKGLTGSPGSPGPDGKTGPPGPAGQDGRPGPPGPPGARGQAGVMGFPGPKGAAGEPGKAGERGVPGPPGAVGPAGKDGEAGAQGPPGPAGPAGERGEQGPAGSPGFQGLPGPAGPPGEAGKPGEQGVPGDLGAPGPSGARGERGFPGERGVQGPPGPAGPRGANGAPGNDGAKGDAGAPGAPGSQGAPGLQGMPGERGAAGLPGPKGDRGDAGPKGADGSPGKDGVRGLTGPIGPPGPAGAPGDKGESGPSGPAGPTGARGAPGDRGEPGPPGPAGFAGPPGADGQPGAKGEPGDAGAKGDAGPPGPAGPAGPPGPIGNVGAPGAKGARGSAGPPGATGFPGAAGRVGPPGPSGNAGPPGPPGPAGKEGGKGPRGETGPAGRPGEVGPPGPPGPAGEKGSPGADGPAGAPGTPGPQGIAGQRGVVGLPGQRGERGFPGLPGPSGEPGKQGPSGASGERGPPGPMGPPGLAGPPGESGREGAPGAEGSPGRDGSPGAKGDRGETGPAGPPGAPGAPGAPGPVGPAGKSGDRGETGPAGPAGPVGPVGARGPAGPQGPRGDKGETGEQGDRGIKGHRGFSGLQGPPGPPGSPGEQGPSGASGPAGPRGPPGSAGAPGKDGLNGLPGPIGPPGPRGRTGDAGPVGPPGPPGPPGPPGPPSAGFDFSFLPQPPQEKAHDGGRYYRAHHHHHH

SEQ ID NO: 8 - DNA, 3216 bp, synthetic construct
  source 1..3216: mol_type = other DNA; note = DNA sequence of gene COL1A1M1 encoding a1 (I) M1 with N-terminal Strep-Tag II tag and C-terminal 6 x His Tag
tggtctcatccacaatttgaaaagcaacttagttatggatacgatgaaaaatccacaggtggaatcagtgttcctggacctatgggtccatcaggtccaagaggtttaccaggacctccaggtgccccaggtccccagggatttcaaggtccaccaggagagcctggtgagccaggagcttctggtccacctggtccccctggaccacctggtcctccaggaaagaatggagatgatggtgaagctggaaaacctggaagacctggagaaagaggaccaccaggaccccagggtgccagaggactgccaggtaccgcaggtctgcctggaatgaaaggtcatagaggattttcaggattagacggtgcaaagggagacgctggacctgcaggaccaaagggtgagccaggaagtccaggagagaatggtgcaccaggacagccaggtccacctggactgcccggtgaaagaggtagacccggagcaccaggaccagcaggtgcaagaggaaatgatggagctacaggtgctgcaggacccccaggtccaacaggaccagccggtcctcccggtttcccaggtgccgttggagcaaaaggtgaagctggtccacagggtccaagaggttctgaaggtccacagggagttagaggagaaccaggaccccctggaccagctggtgcagcaggaccagctggtaaccctggtgctgacggtcagccaggtgctaagggagcaaatggagcaccaggaatagctggtgccccaggatttcccggtgctagaggtccaagtggtccacaaggaccaggaggtccacccggtcccaaaggaaacagtggagaaccaggtgcacccggttcaaagggagatacaggagctaaaggagagcccggtccagtgggtgttcagggaccacccggacctgctggagaggaaggtaaaagaggtgcaagaggtgagccaggaccaacaggtctgcctggtccccctggtgaaagaggtggtccaggtagtagaggatttccaggagctgatggtgttgcaggaccaaagggacccgcaggtgagagaggatcacccggtccagccggaccaaaaggatcaccaggagaagctggtagaccaggagaagctggtctgccaggtgctaaaggattgacaggatcacccggttcacctggtcctgatggaaagacaggacctccaggtcccgctggtcaggacggtagaccaggacccccaggacccccaggtgcaagaggtcaggcaggtgtaatgggtttccccggacctaaaggagcagctggagaacctggtaaagctggagagagaggagtgcctggaccccctggagctgttggtccagcaggaaaggatggtgaggcaggtgcacaaggtccacctggacccgctggacctgcaggtgagagaggagagcaaggtcccgcaggttctccaggttttcagggtttgccaggtccagccggtcctcctggagaggcaggaaagccaggagaacaaggagttccaggagacctgggtgcaccaggaccctctggtgcaagaggagagagaggatttcctggagaaagaggtgtgcagggaccaccaggtcccgccggtccaagaggagcaaatggagcccctggaaatgacggagctaagggtgacgctggtgcaccaggagcaccaggttctcaaggtgctcccggattgcagggtatgcctggagagagaggtgcagctggactgccaggtccaaaaggtgacagaggagacgccggtcctaagggagctgacggttctcctggaaaggacggtgtgagaggtttgacaggaccaataggtccacccggtcctgctggagcccctggagacaaaggtgaatcaggtccttccggtccagccggaccaacaggagcaagaggagcacctggagacagaggagagccaggtcctccaggacctgcaggtttcgctggtcctcccggagcagatggacagccaggagctaagggagaacccggtgacgctggtgctaagggagatgcaggtccaccaggtcctgctggtcctgctggacctcccggaccaataggtaatgttggagcacccggagcaaaaggtgccagaggttccgcaggtcctcccggagcaactggttttccaggagctgccggaagagtgggtccacctggtccttctggaaatgcaggaccaccaggtcctcctggtccagccggaaaggaaggtggaaagggacctagaggagaaacaggtcccgcaggtagacccggtgaggtgggtccacctggtccacccggtccagctggtgagaaaggaagtcctggagcagacggaccagctggtgcccctggtacaccaggaccccaaggaatagctggtcaaagaggtgttgttggtttaccaggtcagagaggagaaagaggttttccaggattaccaggtccctcaggtgagcccggaaaacagggtccctcaggagcaagtggtgaaagaggaccaccaggaccaatgggacctccaggattagctggtccaccaggagaatcaggaagagagggtgctcctggagcagaaggttcaccaggaagagacggttcacccggagccaagggagacagaggtgaaacaggtcccgcaggtccaccaggagcacccggagcccctggtgctccaggacctgtcggaccagcaggaaaatccggtgacagaggtgagactggacccgcaggtcctgctggtcctgttggaccagtgggtgcaagaggaccagcaggtccacaaggtccaagaggtgacaaaggtgagacaggtgagcagggtgacagaggaattaaaggtcacagaggattttcaggactgcagggaccacccggtcctcccggttccccaggagagcaaggtccatccggtgcatccggtccagctggacccagaggaccacctggttctgctggtgcaccaggtaaagatggattgaacggtttgcctggtccaataggacctcctggtccaagaggaagaactggtgacgccggtcccgtcggaccacccggtccaccaggtcccccaggtccacccggaccaccatccgcaggatttgatttctcattccttcctcaacctcctcaagagaaagcacatgatggaggtagatactatagagcccatcaccaccatcatcattaa

SEQ ID NO: 9 - protein, 1076 aa, synthetic construct
  source 1..1076: note = Amino acid sequence of a1 (II) M6 with N-terminal Strep-Tag II tag and C-terminal 6 x His Tag
EFWSHPQFEKQMAGGFDEKAGGAQLGPPQGPPGPPGPPGPPGPAGAPGPQGFQGNPGEPGEPGVSGPPGPPGPPGPPGKPGDDGEAGKPGKAGERGPPGPQGARGFPGTPGLPGVKGHRGYPGLDGAKGEAGAPGVKGESGSPGENGSPGPPGPPGLPGERGRTGPAGAAGARGNDGQPGPAGPPGPVGPAGGPGFPGAPGAKGEAGPTGARGPEGAQGPRGEPGTPGSPGPAGASGNPGTDGIPGAKGSAGAPGIAGAPGFPGPRGPPGPQGATGPLGPKGQTGEPGIAGFKGEQGPKGEPGPAGPQGAPGPAGEEGKRGARGEPGGVGPIGPPGERGAPGNRGFPGQDGLAGPKGAPGERGPSGLAGPKGANGDPGRPGEPGLPGARGLTGRPGDAGPQGKVGPSGAPGEDGRPGPPGPQGARGQPGVMGFPGPKGANGEPGKAGEKGLPGAPGLRGLPGKDGETGAAGPPGPAGPAGERGEQGAPGPSGFQGLPGPPGPPGEGGKPGDQGVPGEAGAPGLVGPRGERGFPGERGSPGAQGLQGPRGLPGTPGTDGPKGASGPAGPPGAQGPPGLQGMPGERGAAGIAGPKGDRGDVGEKGPEGAPGKDGGRGLTGPIGPPGPAGANGEKGEVGPPGPAGSAGARGAPGERGETGPPGPAGFAGPPGADGQPGAKGEQGEAGQKGDAGAPGPQGPSGAPGPQGPTGVTGPKGARGAQGPPGATGFPGAAGRVGPPGSNGNPGPPGPPGPSGKDGPKGARGDSGPPGRAGEPGLQGPAGPPGEKGEPGDDGPSGAEGPPGPQGLAGQRGIVGLPGQRGERGFPGLPGPSGEPGKQGAPGASGDRGPPGPVGPPGLTGPAGEPGREGSPGADGPPGRDGAAGVKGDRGETGAVGAPGAPGPPGSPGPAGPTGKQGDRGEAGAQGPMGPSGPAGARGIQGPQGPRGDKGEAGEPGERGLKGHRGFTGLQGLPGPPGPSGDQGASGPAGPSGPRGPPGPVGPSGKDGANGIPGPIGPPGPRGRSGETGPAGPPGNPGPPGPPGPPGPGIDMSAFAGLGPREKGPDPLQYMRAHHHHHH

SEQ ID NO: 10 - DNA, 3231 bp, synthetic construct
  source 1..3231: mol_type = other DNA; note = DNA sequence of gene COL2A1M6 encoding a1 (II) M6 with N-terminal Strep-Tag II tag and C-terminal 6 x His Tag
gaattctggagtcatcctcaattcgaaaaacaaatggctggtggattcgatgaaaaggctggtggagcccaattaggtcctccacaaggtcctcccggtccacctggtcctcccggtcctccaggtcccgccggtgctcctggaccacagggtttccaaggaaaccccggtgaaccaggtgagcctggtgtttcaggtcctcccggtcctccaggaccacctggaccaccaggaaagcctggtgacgacggagaagctggtaaaccaggaaaggcaggagagagaggtccacctggacctcagggtgccagaggtttcccaggtacccctggtcttcctggtgtcaagggtcatagaggttaccccggtttggatggtgccaagggtgaagccggtgcccctggtgttaagggtgaatcaggaagtcccggtgaaaatggaagtcccggtccacccggtccacctggactgccaggtgagagaggaagaaccggaccagctggtgctgcaggtgctagaggaaatgacggacagcccggaccagccggacctcccggtcctgttgggcccgcaggtggtcctggtttccctggtgctcctggagccaaaggagaagccggacccaccggagccagaggtcccgagggagcacagggacctagaggagaaccaggtacaccaggtagtcccggtcctgctggtgcatcaggaaatcccggaactgacggtattccaggagcaaagggatctgcaggagcaccaggaatagctggtgctcctggatttccaggtcccagaggacctcccggtcctcaaggagcaacaggtcctttgggaccaaaaggtcaaacaggagaaccaggtattgctggattcaaaggagagcaaggtccaaagggagagcccggtcccgcaggtccccaaggagccccaggaccagctggtgaagaaggaaaaagaggagccagaggtgaacctggaggagtaggacctattggtcctcctggtgagagaggtgctcccggaaacagaggttttcctggtcaagatggtctggctggacctaaaggtgctccaggagagagaggaccttcaggacttgctggtccaaaaggtgctaacggagatccaggaagacccggtgaacctggtctgcctggagctagaggattaacaggaagaccaggtgacgcaggtccccagggtaaagtgggtcccagtggtgccccaggtgaagatggaagacctggtcctcccggaccccaaggtgcaagaggtcagcctggagtgatgggatttcctggacccaagggtgctaacggagaacctggaaaagctggtgagaaaggactgcccggtgccccaggtcttagaggtttgccaggtaaagatggagaaacaggagccgcaggaccacccggtccagccggaccagcaggagagagaggtgaacaaggagcacctggtccaagtggttttcagggtcttccaggtccccctggtccaccaggagagggaggtaaaccaggtgaccaaggtgtccctggagaagcaggtgcacccggtcttgtgggtccaagaggtgaaagaggattccctggtgagagaggatctcccggagcccagggacttcaaggtcctagaggtctgccaggtacccctggtacagacggaccaaagggagcatcaggacccgctggacctcccggagcccaaggtcctccaggtttacaaggtatgcctggtgaaagaggtgctgcaggtatagctggaccaaaaggagacagaggtgacgttggtgagaagggtcccgaaggagcccctggaaaagatggtggaagaggattaacaggtcctataggaccacccggtccagccggtgctaatggagaaaaaggagaagtaggtcctccaggtccagcaggatctgcaggtgctagaggtgcccctggagagagaggtgaaacaggaccacctggtccagctggtttcgctggtcccccaggagctgatggacagcccggtgcaaaaggtgaacaaggagaagccggacagaagggagatgctggagcccccggtccacaaggtccctcaggagcaccaggtcctcaaggtccaactggtgtgaccgggccaaagggtgcaagaggagcacagggacctccaggagcaacaggtttcccaggagctgctggtagagtcggtccacccggatctaatggtaaccccggaccaccaggaccacctggaccatctggaaaggatggacccaaaggagcaagaggagattcaggaccacccggaagagcaggagaacctggattacagggtcccgccggtccaccaggagagaaaggagagcccggagatgatggtccctcaggtgcagagggacccccaggaccccaaggtctggcaggtcaaagaggtatagtgggtcttccaggtcaaagaggtgaaagaggatttccaggacttccaggtccttcaggtgaacccggtaaacagggagcccccggagcctcaggtgacagaggtcctccaggaccagtaggacccccaggtttaaccggaccagcaggtgagccaggaagagaaggttctcctggagccgatggacctccaggaagagacggtgcagctggtgttaagggtgacagaggtgaaactggagccgtaggagccccaggtgcccccggaccacccggatcacccggacctgcaggtcctactggtaaacaaggagatagaggagaagccggtgcccagggtcctatgggtccttctggtcctgcaggagcaagaggtatacaaggtccacagggtcccagaggtgacaagggtgaagcaggagaacccggtgagagaggtctgaagggtcatagaggattcaccgggttacagggtttgccaggaccccctggaccaagtggtgaccagggtgcatccggtccagcaggtccttctggaccaagaggtcctcccggtccagttggtccatcaggtaaagacggagccaacggtatcccaggtcccatcggtcctccaggtcctagaggaagaagtggagagactggtcctgctggacctcctggaaaccctggtcctccaggacctccaggtcctccaggtcccggaatagatatgtccgctttcgctggattgggaccaagagagaaaggtcctgaccctcttcaatatatgagagcacaccatcaccatcatcactaa

(19) United States
(12) Patent Application Publication
(10) Pub. No.: US 2025/0034233 A1
(43) Pub. Date: Jan. 30, 2025

(54) RECOMBINANT COLLAGEN AND PREPARATION METHOD AND USE THEREOF

(71) Applicant: JIANGSU TRAUTEC MEDICAL TECHNOLOGY CO., LTD., Changzhou (CN)

(72) Inventors: Jiajia LI, Changzhou (CN); Liping WANG, Changzhou (CN); Huimin LIU, Changzhou (CN); Wenwen JIANG, Changzhou (CN); Chenming QIAN, Changzhou (CN); Pengfei CHENG, Changzhou (CN); Song QIAN, Changzhou (CN)

(73) Assignee: JIANGSU TRAUTEC MEDICAL TECHNOLOGY CO., LTD., Changzhou (CN)

(21) Appl. No.: 18/715,637

(22) PCT Filed: Nov. 23, 2022
(86) PCT No.: PCT/CN2022/133595

§ 371 Date: May 31, 2024

(30) Foreign Application Priority Data
Dec. 3, 2021 (CN) 202111470250.8

(51) Int. Cl. C07K 14/78; C12P 21/02

(52) U.S. Cl. CPC C07K 14/78; C12P 21/02

(57) ABSTRACT

A recombinant collagen and a preparation method and use thereof, and in particular, a full-length collagen a1 chain produced through recombinant expression, and a preparation method and use thereof are provided. When a variant (which is denoted as a1 (I) M1) of a human type I collagen a1 chain (a1 (I) chain) and a variant (which is denoted as a1 (II) M6) of a human type II collagen a1 chain (a1 (II) chain) constructed in the present disclosure are produced through recombinant expression in Pichia pastoris, a main degradation product (a main degradation band) in substantially the same proportion as a target product (a target band) of a full-length a1 chain occurring during the recombinant expression of native full-length a1 (I) chain and a1 (II) chain is eliminated, and a yield of the target product is improved.

### CROSS REFERENCE TO THE RELATED APPLICATIONS

This application n is the national phase entry of International Application No. PCT/CN2022/133595, filed on Nov. 23, 2022, which is based upon and claims priority to Chinese Patent Application No. 202111470250.8, filed on Dec. 3, 2021, the entire contents of which are incorporated herein by reference.

### SEQUENCE LISTING

The instant application contains a Sequence Listing which has been submitted in XML format via EFS-Web and is hereby incorporated by reference in its entirety. Said XML copy is named GBNJIP142_Sequence Listing.xml, created on May 15, 2024, and is 30,500 bytes in size.

### TECHNICAL FIELD

The present disclosure relates to a recombinant collagen and a preparation method and use thereof, and in particular to a full-length collagen a1 chain produced through recombinant expression, and a preparation method and use thereof. The present disclosure belongs to the technical field of collagen expression.

### BACKGROUND

Type I collagen and type II collagen are typical fibrillar collagens in the human body. Type I collagen and type II collagen each are composed of three a peptide chains, and each a peptide chain contains an amino-terminal peptide region, a characteristic (G-X-Y), triplet-repeat sequence region, and a carboxyl-terminal peptide region.

Type I collagen is composed of two a1 chains and one a2 chain. Type I collagen is the most abundant collagen among all types of collagen in the human body, and is found in muscles, skins, arterial walls, and fibrocartilage. Type II collagen is composed of three a1 chains, and is mainly distributed in cartilage tissues, vitreous humors, and corneas. The amount of type II collagen accounts for 90% or more of the total amount of collagen in an adult cartilage matrix. Type II collagen is an essential component for chondrogenic and skeletal pattern formation, skeletal growth, and mature cartilage maintenance.

As an important native biological protein, collagen has unique functional characteristics such as excellent biocompatibility, biological activity, and degradability. Therefore, collagen can be widely used in many fields such as chemical industry, medicine, food, and cosmetics, and is especially suitable for the preparation of various biological devices. Collagen is the most desirable source of biological materials and has promising application prospects.

Commercially available collagen products are mainly collagen extracts obtained by treating animal tissues with acid hydrolysis, alkaline hydrolysis, and enzymatic hydrolysis. However, during the above treatment process, collagen is severely degraded and thus loses its biological activity, and extracted collagen peptides have different lengths, heterogeneous properties, unstable qualities, and potential safety hazards from viral infections such as bovine spongiform encephalopathy and foot-and-mouth disease. In addition, animal-derived collagen has a quite different amino acid sequence from human-derived collagen, and is a heterologous protein, which will lead to immune rejection and allergic symptoms.

The production of a recombinant collagen through genetic engineering can effectively avoid these defects. Among the existing expression methods for recombinant collagen, expression systems such as mammalian cell expression systems, insect cell (baculovirus) expression systems, and transgenic animal and plant expression systems have characteristics such as high costs, low yield, and long cycle, and are mostly used for experiments at a scientific research stage. In large-scale industrial production, prokaryotic (Escherichia coli) expression systems and Pichia pastoris expression systems are mainly used to express human collagen. In Escherichia coli, there is no post-translational modification in the protein, and the protein is expressed intracellularly on a large scale. As a result, the bacterial cells need to be lysed, such that a large number of impurities such as host proteins and native endotoxins and peptidoglycans (as cell wall components) are produced, which can only be removed by a complicated purification process. Because human collagen is a foreign protein for Pichia pastoris after all, the expression of human collagen in Pichia pastoris occupies many intracellular resources (the methanol metabolism pathway relied by the above expression can result in the expression of up to 30% of soluble proteins in the cell), and Pichia pastoris cells will adjust correspondingly in response to a foreign protein. Typically, the recombinant protein is severely degraded in Pichia pastoris. a1 chains of human type I collagen and type II collagen each are a long peptide chain of 1,000 or more amino acids, and thus are prone to degradation.

a1 chains of human type I collagen and type II collagen each contain an amino-terminal peptide, a triple helix region, and a carboxyl-terminal peptide in a mature sequence thereof. A human type I collagen a1 chain (hereinafter canonically referred to as a1 (I)) has a full length of 1,057 amino acids (AA), and a human type II collagen a1 chain (hereinafter canonically referred to as a1 (II)) has a full length of 1,060 AA. There are many studies and patents on the expression of the human a1 (I) chain in Pichia pastoris, and few studies and patents on the expression of the human a1 (II) chain. In most of the existing studies on the expression of a full-length a1 (I) chain and a full-length a1 (II) chain in Pichia pastoris, only a part of the sequence of the a1 (I) chain, rather than the mature full-length sequence of the a1 (I) chain, is expressed. In some published findings, although a full-length a1 (I) chain is expressed, a main degradation product in substantially the same proportion as a target product is produced during the expression, and thus a main degradation band (the main degradation product) in substantially the same proportion as a target band (the target product) of the full-length a1 chain is presented during sodium dodecyl sulfate-polyacrylamide gel electrophoresis (SDS-PAGE). Such degradation results in reduced yield of the full-length a1 chain expressed. In addition, because the main degradation product has similar correlated properties to the full-length a1 chain, two-step tandem affinity purification is required to obtain a high-purity single full-length a1 chain product, which increases the complexity of the purification process and increases purification costs accordingly. Therefore, an important challenge for the production of a recombinant collagen in Pichia pastoris is to keep the full-length peptide chain intact and reduce the degradation, while maintaining the biological activity of the collagen unchanged.

### SUMMARY

An objective of the present disclosure is to overcome some technical problems in the prior art, and to provide a full-length collagen a1 chain produced through recombinant expression in Pichia pastoris, and a preparation method and use thereof. Compared with native full-length a1 (1) and a1 (II) chains, when a variant of a recombinant human type I collagen a1 chain (which is denoted as a1 (I) M1) and a variant of a recombinant human type II collagen a1 chain (which is denoted as a1 (II) M6) according to the present disclosure are expressed in Pichia pastoris, a main degradation band (a main degradation product) in substantially the same proportion as a target band (a target product) of a full-length a1 chain is eliminated, and a yield of the target product is improved. The variants according to the present disclosure have similar physical and chemical characteristics and identical biological activity to the native full-length collagen a1 (I) and a1 (II) chains and commercially available human collagens expressed in Pichia pastoris, and thus both have application values in the field of biomedical materials.

To achieve the above objective, the present disclosure adopts the following technical solutions.

The present disclosure provides a recombinant collagen a1 chain, where the recombinant collagen a1 chain is a1 (I) M1 or a1 (II) M6; and the a1 (I) M1 is obtained through an amino acid mutation in a native full-length amino acid sequence of a human type I collagen a1 chain, and the a1 (II) M6 is obtained through an amino acid mutation in a native full-length amino acid sequence of a human type II collagen a1 chain.

Preferably, the a1 (I) M1 has 4 amino acid mutation sites, and the a1 (II) M6 has 9 amino acid mutation sites.

Preferably, the human type I collagen a1 chain is set forth in SEQ ID NO: 1; and the amino acid mutation sites include M at position 106, R at position 109, M at position 190, and R at position 193, and specifically, the amino acid mutation sites are mutated to P.

The human type II collagen a1 chain is set forth in SEQ ID NO: 4; and the amino acid mutation sites include V at position 67, M at position 68, M at position 72, M at position 75, R at position 78, M at position 108, R at position 111, M at position 162, and R at position 165, and specifically, the amino acid mutation sites are mutated to P.

Further, the a1 (I) M1 has an amino acid sequence as set forth in SEQ ID NO: 2, and the a1 (II) M6 has an amino acid sequence as set forth in SEQ ID NO: 5.

In the present disclosure, when the amino acid mutations at the amino acid mutation sites of the amino acid sequence are changed to some extent, that is, an amino acid(s) at the same amino acid mutation site(s) of the amino acid sequence is mutated to a different amino acid(s), technical effects similar to those of the present disclosure can also be obtained. When one or more of the amino acid mutation sites is/are changed, technical effects similar to those of the present disclosure may also be obtained.

The present disclosure also provides a nucleotide encoding the recombinant collagen a1 chain, where a sequence of the nucleotide encoding the recombinant collagen a1 chain includes a nucleotide sequence encoding the a1 (I) M1 or a nucleotide sequence encoding the a1 (II) M6.

Further, the nucleotide sequence encoding the a1 (I) M1 is set forth in SEQ ID NO: 3; and the nucleotide sequence encoding the a1 (II) M6 is set forth in SEQ ID NO: 6.

The present disclosure also provides a recombinant expression vector containing the nucleotide encoding the recombinant collagen a1 chain.

The present disclosure also provides an engineered strain constructed with the recombinant expression vector, where the engineered strain carries the recombinant expression vector or expresses the recombinant collagen a1 chain.

A host strain for the engineered strain is preferably Pichia pastoris. The engineered strain was deposited in the China General Microbiological Culture Collection Center (CGMCC) located at NO. 1, West Beichen Road, Chaoyang District, Beijing, China on Mar. 11, 2021, with an accession number of CGMCC NO. 21891 or CGMCC NO. 21892 and a taxonomic name of Pichia pastoris. The engineered strain with the accession number of CGMCC NO. 21891 expresses the recombinant collagen a1 chain a1 (I) M1, and the engineered strain with the accession number of CGMCC NO. 21892 expresses the recombinant collagen a1 chain a1 (II) M6.

It should be noted that the host strain of the present disclosure is not limited to Pichia pastoris. According to the method of the present disclosure, the recombinant collagen a1 chain can be produced by secretory expression in Pichia pastoris or other yeast species, and technical effects similar to those of the present disclosure can theoretically be obtained.

The present disclosure also provides a use of the recombinant expression vector or the engineered strain in an expression of the recombinant collagen a1 chain.

The present disclosure also provides a preparation method of the recombinant collagen a1 chain, including:

(1) Synthesizing a nucleotide sequence encoding the recombinant collagen a1 chain;

where respective amino acids at amino acid mutation sites of an amino acid sequence of a native collagen a1 chain are mutated. 4 amino acids of a type I collagen a1 chain are mutated to obtain a1 (I) M1, and 9 amino acids of a type II collagen a1 chain are mutated to obtain a1 (II) M6. Affinity purification tags are added to an amino terminus and a carboxyl terminus of the amino acid sequence, and then a DNA sequence encoding a1 (I) M1 or a1 (II) M6 is synthesized, such that the DNA sequence includes bispecific affinity purification tags, which facilitates the immunological antibody detection based on the two tag sequences.

Detection results show that, when the a1 (I) M1 or a1 (II) M6 is expressed in Pichia pastoris, a main degradation band (a main degradation product) in substantially the same proportion as a target band (a target product) of a full-length a1 chain produced when the full-length a1 (I) chain or full-length a1 (II) chain is expressed in Pichia pastoris is eliminated.

Compared with the original native sequences, mutated amino acids in the a1 (I) M1 and a1 (II) M6 are located in a characteristic (G-X-Y), triplet-repeat sequence region, but all are amino acids located on X and Y, such that structural characteristics of an amino acid sequence of the (G-X-Y), triplet-repeat sequence in collagen do not change. In addition, the a1 (I) M1 and a1 (II) M6 still maintain physical and chemical characteristics and biological activities similar to those of the original collagen.

(2) Constructing a recombinant expression vector;

where the synthesized DNA sequences are ligated to expression vectors pPIC9K to construct a recombinant expression vector pPIC9K-COLIAIMI expressing the recombinant collagen a1 (I) M1 and a recombinant expression vector pPIC9K-COL2AIM6 expressing the recombinant collagen a1 (II) M6, respectively.

(3) Constructing recombinant engineered strains for an inducible expression, and screening the recombinant engineered strains;

where the recombinant expression vector is linearized with Sac I and then electrotransformed into Pichia pastoris competent cells, the electrotransformed Pichia pastoris competent cells are transferred to a Minimal Dextrose (MD) plate for primary screening and then further screened on Yeast Extract Peptone Dextrose (YPD) plates with different concentrations of G418, and then colonies are picked and inoculated into a Buffered Glycerol-complex Medium (BMGY medium) and subjected to inducible expression in a Buffered Methanol-complex Medium (BMMY medium). An engineered strain with a high expression level is screened out.

The engineered strain with the high expression level screened out is Pichia pastoris, and has an accession number of CGMCC NO. 21891 or CGMCC NO. 21892.

(4) Cultivating for high-density fermentation;

where the engineered strain with the high expression level identified through protein expression is cultivated in a fermentation tank for a high-density fermentation to obtain a fermentation supernatant.

(5) Purifying the fermentation supernatant to obtain a protein;

where the fermentation supernatant is purified by one-step cation exchange chromatography and then lyophilized to obtain a high-purity collagen a1 (I) M1 or a1 (II) M6.

The obtained recombinant collagen a1 (I) M1 and recombinant collagen a1 (II) M6 expressed in Pichia pastoris according to the present disclosure are analyzed through protein property characterization and in vitro experiments. The recombinant collagen a1 (I) M1 and recombinant collagen «1 (II) M6 obtained in the present disclosure have structural characteristics of recombinant collagen, have cell adhesion activities, and are substantially consistent with the commercially available human collagens. More importantly, the two variant proteins of the present disclosure have similar or identical structural characteristics and cell adhesion activities compared with the unmutated original collagen.

The present disclosure also provides a composition including the recombinant collagen a1 chain or a collagen a1 chain prepared by the preparation method described above.

The present disclosure also provides an article including the recombinant collagen a1 chain or a collagen a1 chain prepared by the preparation method described above or the composition described above. The article includes, but is not limited to, a drug, a pharmaceutical composition, a medical device, a biological material, a tissue-engineered product, a cosmetic, or a health product.

Further, the article includes a material for providing an adhesion, a support, and a growth and migration space for a cell or a material serving as a channel for delivering a nutrient and a metabolite.

Further, the article is a collagen hydrogel.

The present disclosure also provides a use of the recombinant collagen a1 chain, the nucleotide, the recombinant expression vector, the engineered strain, or the composition in the manufacture of a product, where the product includes, but is not limited to, a drug, a medical device, a biological material, a tissue-engineered product, a cosmetic, or a health product.

The present disclosure also provides a use of the recombinant collagen a1 chain, the nucleotide, the recombinant expression vector, the engineered strain, or the composition in the manufacture of a product for promoting wound healing or tissue regeneration. Further, the product is a collagen hydrogel.

### The Present Disclosure has the Following Advantages.

(1) In the collagen a1 chain variants of the present disclosure, a proportion of the amino acid mutation sites in the original native sequence is very small (the proportion of mutated amino acids is lower than 1%, and a homology between amino acid sequences before and after mutation is higher than 99%), such that the complete recombinant a1 chain collagen is obtained without changing the properties (physical and chemical characteristics and biological activity) of the original protein itself, and the collagen a1 chain variants of the present disclosure have the same properties and biological activities as a recombinant protein of a native sequence when prepared into related products.

The a1 (I) M1 and a1 (II) M6 have physical and chemical characteristics and biological activities similar to those of a1 (I) and a1 (II), respectively, and both have application values in the field of biomedical materials. In the present disclosure, it is found through cell adhesion experiments on a1 (I) M1, a1 (II) M6, a1 (I), and a1 (II) that, there is no significant difference in the cell adhesion activity between a1 (I) and a1 (I) M1 and between a1 (II) M6 and a1 (II), and the cell adhesion activities of the a1 (I) M1 and a1 (II) M6 are substantially consistent with those of the commercially available human collagens. Collagen hydrogels are prepared from collagens a1 (I) M1, a1 (II) M6, a1 (I), and a1 (II) expressed in Pichia pastoris, and hydromechanical characteristics of the collagen hydrogels are tested. There is no significant difference in the viscosity, elasticity modulus, and swelling degree between the collagen hydrogel prepared from a1 (I) and the collagen hydrogel prepared from a1 (I) M1 and between the collagen hydrogel prepared from a1 (II) M6 and the collagen hydrogel prepared from a1 (II). Lyophilized collagen hydrogels are scanned by scanning electron microscopy (SEM). These collagen hydrogels all have a porous network structure and a pore size ranging from 100 μm to 200 μm, and have the potential to be applied in the field of biomedical materials. The four collagen hydrogels each are co-cultivated with NIH/3T3 cells in vitro. After Calcein Acetoxymethyl Ester (Calcein AM) is added, viable cells exhibiting green fluorescence that adhere to the collagen hydrogel and grow in the collagen hydrogel can be detected. After methylthiazolyldiphenyl-tetrazolium bromide (MTT) is added, blue-purple crystals produced by viable cells that adhere to the collagen hydrogel and migrate and grow into the collagen hydrogel can be observed.

(2) In the present disclosure, expressed proteins are identified by SDS-PAGE and Western blot (WB). Identification results show that, compared with the full-length a1 (I) chain and full-length a1 (II) chain, when the a1 (I) M1 and a1 (II) M6 are expressed in Pichia pastoris, a main degradation band (a main degradation product) in substantially the same proportion as a target band (a target product) of a full-length a1 chain is eliminated, and the yield of the target product is improved. In addition, a high-density fermentation experiment is performed in a fermentation tank, and fermentation products are detected by SDS-PAGE. It can be found that a1 (I) M1 and a1 (II) M6 each can still maintain the integrity of the target band and do not lead to a main degradation band under high-density fermentation conditions, while the recombinant human a1 (I) and a1 (II) produced by fermentation under the same high-density fermentation conditions lead to obvious main degradation bands.

Moreover, for the recombinant collagen of the present disclosure, the fermentation supernatant only needs to be purified by one-step cation exchange chromatography and then lyophilized to obtain the lyophilized high-purity a1 (I) M1 or a1 (II) M6 collagen sponge. The collagen sponge is detected by SDS-PAGE, and there is mainly a single band of the target product, and no main degradation band, indicating that the high-purity target product is obtained. In the present disclosure, the cost of purification is reduced. However, for a1 (I) and a1 (II), after the fermentation supernatant is purified by one-step cation exchange chromatography, a purified protein product that is a mixture of a target band (a target product, a full-length a1 chain) and a main degradation band (a main degradation product) can only be obtained. Thus, two-step affinity chromatography is required to obtain the high-purity collagen a1 (I) or a1 (II).

### BRIEF DESCRIPTION OF THE DRAWINGS

FIG. 1 shows differences between amino acid sequences of a1 (I) M1 and a1 (I), where amino acids in bold and gray background indicate difference sites.

FIG. 2 shows differences between amino acid sequences of a1 (II) M6 and a1 (II), where amino acids in bold and gray background indicate difference sites.

FIG. 3 is a map of a pPIC9K-COLIAIMI vector.

FIG. 4 is a map of a pPIC9K-COL2AIM6 vector.

FIG. 5 shows an SDS-PAGE pattern of supernatants produced after inducible expression of a1 (I) M1 and a1 (II) M6 collagens for 24 h.

FIGS. 6A-6B show WB patterns of supernatants produced after inducible expression of a1 (I) M1 and a1 (II) M6 collagens for 24 h, where FIG. 6A is a WB pattern with an anti-6×His Tag antibody and FIG. 6B is a WB pattern with an anti-Strep-Tag II antibody.

FIG. 7A, FIG. 7B, FIG. 7C, and FIG. 7D show mass spectrometry results of target bands and main degradation bands in SDS-PAGE results of a1 (I) and a1 (II) collagens.

FIG. 8A and FIG. 8B show mass spectrometry results of target bands in SDS-PAGE results of a1 (I) M1 and a1 (II) M6 collagens.

FIGS. 9A-9B show SDS-PAGE patterns of fermentation supernatants produced after inducible expression of a1 (I) M1, a1 (II) M6, a1 (I), and a1 (II) collagens for 48 h.

FIGS. 10A-10B show SDS-PAGE patterns of purified and lyophilized a1 (I) M1, a1 (II) M6, a1 (I), and a1 (II) sponges.

FIG. 11A and FIG. 11B show Fourier transform infrared (FT-IR) spectroscopy results of a1 (1) and a1 (II) collagens.

FIG. 12A and FIG. 12B show FT-IR spectroscopy results of a1 (I) M1 and a1 (II) M6 collagens.

FIG. 13 shows cell adhesion activity results of a1 (I) M1, a1 (II) M6, a1 (I), and a1 (II) collagens.

FIGS. 14A-14D show SEM images of surfaces of lyophilized a1 (I) and a1 (I) M1 collagen hydrogels (FIGS. 14A-14B show the a1 (I) collagen hydrogel and FIGS. 14C-14D show the a1 (I) M1 collagen hydrogel).

FIGS. 15A-15D show SEM images of surfaces of lyophilized a1 (II) and a1 (II) M6 collagen hydrogels (FIGS. 15A-15B show the a1 (II) collagen hydrogel and FIGS. 15C-15D show the a1 (II) M6 collagen hydrogel).

FIGS. 16A-16F show results of adherent growth of NIH/3T3 cells in a1 (I) and a1 (I) M1 collagen hydrogels, where FIGS. 16A-16C show the following respectively: NIH/3T3 cells growing adherently on the a1 (I) collagen hydrogel (the image being taken with a brightfield microscope), NIH/3T3 cells growing adherently on the a1 (I) collagen hydrogel (stained with Calcein AM, bright parts show cells exhibiting green fluorescence, and the image being taken with a fluorescence microscope), and blue-purple crystals produced by NIH/3T3 cells growing in the a1 (I) collagen hydrogel (stained with MTT, dark parts show the crystals, and the image being taken with a brightfield microscope); and

FIGS. 16D-16F show the following respectively: NIH/3T3 cells growing adherently on the a1 (I) M1 collagen hydrogel (the image being taken with a brightfield microscope), NIH/3T3 cells growing adherently on the a1 (I) M1 collagen hydrogel (stained with Calcein AM, bright parts show cells exhibiting green fluorescence, and the image being taken with a fluorescence microscope), and blue-purple crystals produced by NIH/3T3 cells growing in the a1 (I) M1 collagen hydrogel (stained with MTT, dark parts show the crystals, and the image being taken with a brightfield microscope).

FIGS. 17A-17F show results of adherent growth of NIH/3T3 cells in a1 (II) and a1 (II) M6 collagen hydrogels,

where FIGS. 17A-17C show the following respectively: NIH/3T3 cells growing adherently on the a1 (II) collagen hydrogel (the image being taken with a brightfield microscope), NIH/3T3 cells growing adherently on the a1 (II) collagen hydrogel (stained with Calcein AM, bright parts show cells exhibiting green fluorescence, and the image being taken with a fluorescence microscope), and blue-purple crystals produced by NIH/3T3 cells growing in the a1 (II) collagen hydrogel (stained with MTT, dark parts show the crystals, and the image being taken with a brightfield microscope); and

FIGS. 17D-17F show the following respectively: NIH/3T3 cells growing adherently on the a1 (II) M6 collagen hydrogel (the image being taken with a brightfield microscope), NIH/3T3 cells growing adherently on the a1 (II) M6 collagen hydrogel (stained with Calcein AM, bright parts show cells exhibiting green fluorescence, and the image being taken with a fluorescence microscope), and blue-purple crystals produced by NIH/3T3 cells growing in the a1 (II) M6 collagen hydrogel (stained with MTT, dark parts show the crystals, and the image being taken with a brightfield microscope).

### DETAILED DESCRIPTION OF THE EMBODIMENTS

In order to make those skilled in the art better understand the technical solutions of the present disclosure, the preferred examples of the present disclosure are described in detail below, but the following examples do not limit the protection scope of the present disclosure.

In the examples of the present disclosure, those not described in detail are all implemented by a conventional molecular biology experimental method; and processes such as polymerase chain reaction (PCR), enzyme digestion, ligation, and codon optimization involved in the examples can all be understood and easily implemented by those skilled in the art according to product instructions or basic knowledge in the art, and thus will not be described in detail.

### Example 1 Design and Synthesis of Amino Acid Sequences

With reference to positions 162 to 1218 (PRO_0000005720) of a P02452-1 (https://www.uniprot.org/uniprot/P02452) sequence in the Uniprot database, an amino acid sequence of a human type I collagen a1 chain (denoted as a1 (I)) is an amino acid sequence of a human type I collagen a1 chain in a mature form, and does not include regions that would be processed and cleaved in a1 (I) proprotein, such as a signal peptide, a C-terminal propeptide, and an N-terminal propeptide, and is set forth in SEQ ID NO: 1.

SEQ ID NO: 1:
QLSYGYDEKSTGGISVPGPMGPSGPRGLPGPPGAPGPQGFQGPPGEPGEPGASGPM
GPRGPPGPPGKNGDDGEAGKPGRPGERGPPGPQGARGLPGTAGLPGMKGHRGFSGLDG
AKGDAGPAGPKGEPGSPGENGAPGQMGPRGLPGERGRPGAPGPAGARGNDGATGAAG
PPGPTGPAGPPGFPGAVGAKGEAGPQGPRGSEGPQGVRGEPGPPGPAGAAGPAGNPGA
DGQPGAKGANGAPGIAGAPGFPGARGPSGPQGPGGPPGPKGNSGEPGAPGSKGDTGAK
GEPGPVGVQGPPGPAGEEGKRGARGEPGPTGLPGPPGERGGPGSRGFPGADGVAGPKGP
AGERGSPGPAGPKGSPGEAGRPGEAGLPGAKGLTGSPGSPGPDGKTGPPGPAGQDGRPG
PPGPPGARGQAGVMGFPGPKGAAGEPGKAGERGVPGPPGAVGPAGKDGEAGAQGPPG
PAGPAGERGEQGPAGSPGFQGLPGPAGPPGEAGKPGEQGVPGDLGAPGPSGARGERGFP
GERGVQGPPGPAGPRGANGAPGNDGAKGDAGAPGAPGSQGAPGLQGMPGERGAAGLP
GPKGDRGDAGPKGADGSPGKDGVRGLTGPIGPPGPAGAPGDKGESGPSGPAGPTGARG
APGDRGEPGPPGPAGFAGPPGADGQPGAKGEPGDAGAKGDAGPPGPAGPAGPPGPIGN
VGAPGAKGARGSAGPPGATGFPGAAGRVGPPGPSGNAGPPGPPGPAGKEGGKGPRGET
GPAGRPGEVGPPGPPGPAGEKGSPGADGPAGAPGTPGPQGIAGQRGVVGLPGQRGERGF
PGLPGPSGEPGKQGPSGASGERGPPGPMGPPGLAGPPGESGREGAPGAEGSPGRDGSPG
AKGDRGETGPAGPPGAPGAPGAPGPVGPAGKSGDRGETGPAGPAGPVGPVGARGPAGP
QGPRGDKGETGEQGDRGIKGHRGFSGLQGPPGPPGSPGEQGPSGASGPAGPRGPPGSAG
APGKDGLNGLPGPIGPPGPRGRTGDAGPVGPPGPPGPPGPPGPPSAGFDFSFLPQPPQEKA
HDGGRYYRA.

A long-term experimental research was performed to obtain a variant of a recombinant human type I collagen a1 chain, which was denoted as a1 (I) M1. Compared with the amino acid sequence of a1 (I), 4 amino acids were mutated to proline (Pro, abbreviated as P) in a1 (1) M1. That is, M at position 106, R at position 109, M at position 190, and R at position 193 in the amino acid sequence set forth in SEQ ID NO: 1 all were mutated to P, and the remaining amino acids in the amino acid sequence remained unchanged. A homology of a1 (I) M1 with a1 (I) was 99.6%.

The mutated amino acid sequence (a1 (I) M1) has a full length of 1,057 AA, and is set forth in SEQ ID NO: 2.

SEQ ID NO: 2:
QLSYGYDEKSTGGISVPGPMGPSGPRGLPGPPGAPGPQGFQGPPGEPGEPGASGPPG
PPGPPGPPGKNGDDGEAGKPGRPGERGPPGPQGARGLPGTAGLPGMKGHRGFSGLDGA
KGDAGPAGPKGEPGSPGENGAPGQPGPPGLPGERGRPGAPGPAGARGNDGATGAAGPP
GPTGPAGPPGFPGAVGAKGEAGPQGPRGSEGPQGVRGEPGPPGPAGAAGPAGNPGADG
QPGAKGANGAPGIAGAPGFPGARGPSGPQGPGGPPGPKGNSGEPGAPGSKGDTGAKGE
PGPVGVQGPPGPAGEEGKRGARGEPGPTGLPGPPGERGGPGSRGFPGADGVAGPKGPA
GERGSPGPAGPKGSPGEAGRPGEAGLPGAKGLTGSPGSPGPDGKTGPPGPAGQDGRPGP
PGPPGARGQAGVMGFPGPKGAAGEPGKAGERGVPGPPGAVGPAGKDGEAGAQGPPGP
AGPAGERGEQGPAGSPGFQGLPGPAGPPGEAGKPGEQGVPGDLGAPGPSGARGERGFP
GERGVQGPPGPAGPRGANGAPGNDGAKGDAGAPGAPGSQGAPGLQGMPGERGAAGLP
GPKGDRGDAGPKGADGSPGKDGVRGLTGPIGPPGPAGAPGDKGESGPSGPAGPTGARG
APGDRGEPGPPGPAGFAGPPGADGQPGAKGEPGDAGAKGDAGPPGPAGPAGPPGPIGN
VGAPGAKGARGSAGPPGATGFPGAAGRVGPPGPSGNAGPPGPPGPAGKEGGKGPRGET
GPAGRPGEVGPPGPPGPAGEKGSPGADGPAGAPGTPGPQGIAGQRGVVGLPGQRGERGF
PGLPGPSGEPGKQGPSGASGERGPPGPMGPPGLAGPPGESGREGAPGAEGSPGRDGSPG
AKGDRGETGPAGPPGAPGAPGAPGPVGPAGKSGDRGETGPAGPAGPVGPVGARGPAGP
QGPRGDKGETGEQGDRGIKGHRGFSGLOGPPGPPGSPGEQGPSGASGPAGPRGPPGSAG
APGKDGLNGLPGPIGPPGPRGRTGDAGPVGPPGPPGPPGPPGPPSAGFDFSFLPQPPQEKA
HDGGRYYRA.

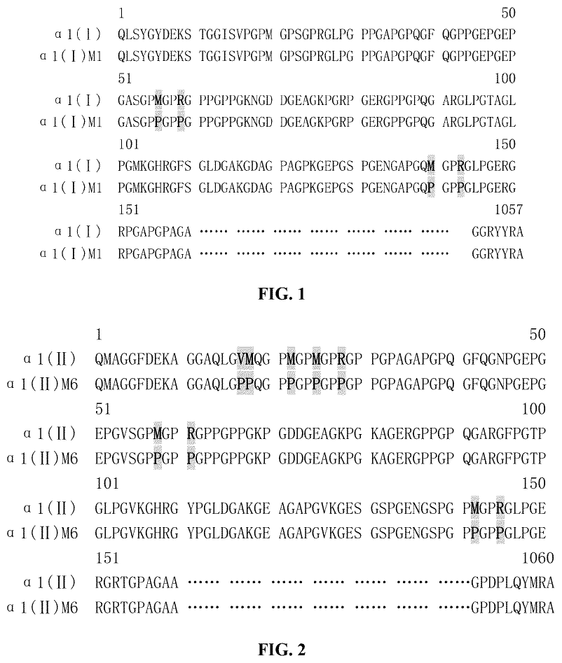

Differences between the amino acid sequences of a1 (I) M1 and a1 (D) are shown by amino acids in bold and gray background in FIG. 1.

A DNA sequence of a gene (denoted as COLIAIMI) encoding the a1 (1) M1 set forth in SEQ ID NO: 2 is set forth in SEQ ID NO: 3.

SEQ ID NO: 3:
CAACTTAGTTATGGATACGATGAAAAATCCACAGGTGGAATCAGTGTTCCTGGA
CCTATGGGTCCATCAGGTCCAAGAGGTTTACCAGGACCTCCAGGTGCCCCAGGTCCC
CAGGGATTTCAAGGTCCACCAGGAGAGCCTGGTGAGCCAGGAGCTTCTGGTCCACC
TGGTCCCCCTGGACCACCTGGTCCTCCAGGAAAGAATGGAGATGATGGTGAAGCTG
GAAAACCTGGAAGACCTGGAGAAAGAGGACCACCAGGACCCCAGGGTGCCAGAGG
ACTGCCAGGTACCGCAGGTCTGCCTGGAATGAAAGGTCATAGAGGATTTTCAGGATT
AGACGGTGCAAAGGGAGACGCTGGACCTGCAGGACCAAAGGGTGAGCCAGGAAGT
CCAGGAGAGAATGGTGCACCAGGACAGCCAGGTCCACCTGGACTGCCCGGTGAAAG
AGGTAGACCCGGAGCACCAGGACCAGCAGGTGCAAGAGGAAATGATGGAGCTACA
GGTGCTGCAGGACCCCCAGGTCCAACAGGACCAGCCGGTCCTCCCGGTTTCCCAGGT
GCCGTTGGAGCAAAAGGTGAAGCTGGTCCACAGGGTCCAAGAGGTTCTGAAGGTCC
ACAGGGAGTTAGAGGAGAACCAGGACCCCCTGGACCAGCTGGTGCAGCAGGACCA
GCTGGTAACCCTGGTGCTGACGGTCAGCCAGGTGCTAAGGGAGCAAATGGAGCACC
AGGAATAGCTGGTGCCCCAGGATTTCCCGGTGCTAGAGGTCCAAGTGGTCCACAAG
GACCAGGAGGTCCACCCGGTCCCAAAGGAAACAGTGGAGAACCAGGTGCACCCGGT
TCAAAGGGAGATACAGGAGCTAAAGGAGAGCCCGGTCCAGTGGGTGTTCAGGGACC
ACCCGGACCTGCTGGAGAGGAAGGTAAAAGAGGTGCAAGAGGTGAGCCAGGACCA
ACAGGTCTGCCTGGTCCCCCTGGTGAAAGAGGTGGTCCAGGTAGTAGAGGATTTCCA
GGAGCTGATGGTGTTGCAGGACCAAAGGGACCCGCAGGTGAGAGAGGATCACCCGG
TCCAGCCGGACCAAAAGGATCACCAGGAGAAGCTGGTAGACCAGGAGAAGCTGGT
CTGCCAGGTGCTAAAGGATTGACAGGATCACCCGGTTCACCTGGTCCTGATGGAAA
GACAGGACCTCCAGGTCCCGCTGGTCAGGACGGTAGACCAGGACCCCCAGGACCCC
CAGGTGCAAGAGGTCAGGCAGGTGTAATGGGTTTCCCCGGACCTAAAGGAGCAGCT
GGAGAACCTGGTAAAGCTGGAGAGAGAGGAGTGCCTGGACCCCCTGGAGCTGTTGG
TCCAGCAGGAAAGGATGGTGAGGCAGGTGCACAAGGTCCACCTGGACCCGCTGGAC
CTGCAGGTGAGAGAGGAGAGCAAGGTCCCGCAGGTTCTCCAGGTTTTCAGGGTTTG
CCAGGTCCAGCCGGTCCTCCTGGAGAGGCAGGAAAGCCAGGAGAACAAGGAGTTCC
AGGAGACCTGGGTGCACCAGGACCCTCTGGTGCAAGAGGAGAGAGAGGATTTCCTG
GAGAAAGAGGTGTGCAGGGACCACCAGGTCCCGCCGGTCCAAGAGGAGCAAATGG
AGCCCCTGGAAATGACGGAGCTAAGGGTGACGCTGGTGCACCAGGAGCACCAGGTT
CTCAAGGTGCTCCCGGATTGCAGGGTATGCCTGGAGAGAGAGGTGCAGCTGGACTG
CCAGGTCCAAAAGGTGACAGAGGAGACGCCGGTCCTAAGGGAGCTGACGGTTCTCC
TGGAAAGGACGGTGTGAGAGGTTTGACAGGACCAATAGGTCCACCCGGTCCTGCTG
GAGCCCCTGGAGACAAAGGTGAATCAGGTCCTTCCGGTCCAGCCGGACCAACAGGA
GCAAGAGGAGCACCTGGAGACAGAGGAGAGCCAGGTCCTCCAGGACCTGCAGGTTT
CGCTGGTCCTCCCGGAGCAGATGGACAGCCAGGAGCTAAGGGAGAACCCGGTGACG
CTGGTGCTAAGGGAGATGCAGGTCCACCAGGTCCTGCTGGTCCTGCTGGACCTCCCG
GACCAATAGGTAATGTTGGAGCACCCGGAGCAAAAGGTGCCAGAGGTTCCGCAGGT
CCTCCCGGAGCAACTGGTTTTCCAGGAGCTGCCGGAAGAGTGGGTCCACCTGGTCCT
TCTGGAAATGCAGGACCACCAGGTCCTCCTGGTCCAGCCGGAAAGGAAGGTGGAAA
GGGACCTAGAGGAGAAACAGGTCCCGCAGGTAGACCCGGTGAGGTGGGTCCACCTG
GTCCACCCGGTCCAGCTGGTGAGAAAGGAAGTCCTGGAGCAGACGGACCAGCTGGT
GCCCCTGGTACACCAGGACCCCAAGGAATAGCTGGTCAAAGAGGTGTTGTTGGTTTA
CCAGGTCAGAGAGGAGAAAGAGGTTTTCCAGGATTACCAGGTCCCTCAGGTGAGCC
CGGAAAACAGGGTCCCTCAGGAGCAAGTGGTGAAAGAGGACCACCAGGACCAATG
GGACCTCCAGGATTAGCTGGTCCACCAGGAGAATCAGGAAGAGAGGGTGCTCCTGG
AGCAGAAGGTTCACCAGGAAGAGACGGTTCACCCGGAGCCAAGGGAGACAGAGGT
GAAACAGGTCCCGCAGGTCCACCAGGAGCACCCGGAGCCCCTGGTGCTCCAGGACC
TGTCGGACCAGCAGGAAAATCCGGTGACAGAGGTGAGACTGGACCCGCAGGTCCTG
CTGGTCCTGTTGGACCAGTGGGTGCAAGAGGACCAGCAGGTCCACAAGGTCCAAGA
GGTGACAAAGGTGAGACAGGTGAGCAGGGTGACAGAGGAATTAAAGGTCACAGAG
GATTTTCAGGACTGCAGGGACCACCCGGTCCTCCCGGTTCCCCAGGAGAGCAAGGT
CCATCCGGTGCATCCGGTCCAGCTGGACCCAGAGGACCACCTGGTTCTGCTGGTGCA
CCAGGTAAAGATGGATTGAACGGTTTGCCTGGTCCAATAGGACCTCCTGGTCCAAGA
GGAAGAACTGGTGACGCCGGTCCCGTCGGACCACCCGGTCCACCAGGTCCCCCAGG
TCCACCCGGACCACCATCCGCAGGATTTGATTTCTCATTCCTTCCTCAACCTCCTCAA
GAGAAAGCACATGATGGAGGTAGATACTATAGAGCC.

With reference to positions 182 to 1241 (PRO_0000005730) of a P02458 (https://www.uniprot.org/uniprot/P02458) sequence in the Uniprot database, an amino acid sequence of a human type II collagen a1 chain (denoted as a1 (I)) is an amino acid sequence of a human type II collagen a1 chain in a mature form, and does not include regions that would be processed and cleaved in a1 (II) proprotein, such as a signal peptide, a C-terminal propeptide, and an N-terminal propeptide, and is set forth in SEQ ID NO: 4.

SEQ ID NO: 4:
QMAGGFDEKAGGAQLGVMQGPMGPMGPRGPPGPAGAPGPQGFQGNPGEPGEPG
VSGPMGPRGPPGPPGKPGDDGEAGKPGKAGERGPPGPQGARGFPGTPGLPGVKGHRGY
PGLDGAKGEAGAPGVKGESGSPGENGSPGPMGPRGLPGERGRTGPAGAAGARGNDGQ
PGPAGPPGPVGPAGGPGFPGAPGAKGEAGPTGARGPEGAQGPRGEPGTPGSPGPAGASG
NPGTDGIPGAKGSAGAPGIAGAPGFPGPRGPPGPQGATGPLGPKGQTGEPGIAGFKGEQG
PKGEPGPAGPQGAPGPAGEEGKRGARGEPGGVGPIGPPGERGAPGNRGFPGQDGLAGP
KGAPGERGPSGLAGPKGANGDPGRPGEPGLPGARGLTGRPGDAGPQGKVGPSGAPGED
GRPGPPGPQGARGQPGVMGFPGPKGANGEPGKAGEKGLPGAPGLRGLPGKDGETGAA
GPPGPAGPAGERGEQGAPGPSGFQGLPGPPGPPGEGGKPGDQGVPGEAGAPGLVGPRGE
RGFPGERGSPGAQGLQGPRGLPGTPGTDGPKGASGPAGPPGAQGPPGLQGMPGERGAA
GIAGPKGDRGDVGEKGPEGAPGKDGGRGLTGPIGPPGPAGANGEKGEVGPPGPAGSAG
ARGAPGERGETGPPGPAGFAGPPGADGQPGAKGEQGEAGQKGDAGAPGPQGPSGAPGP
QGPTGVTGPKGARGAQGPPGATGFPGAAGRVGPPGSNGNPGPPGPPGPSGKDGPKGAR
GDSGPPGRAGEPGLQGPAGPPGEKGEPGDDGPSGAEGPPGPQGLAGQRGIVGLPGQRGE
RGFPGLPGPSGEPGKQGAPGASGDRGPPGPVGPPGLTGPAGEPGREGSPGADGPPGRDG
AAGVKGDRGETGAVGAPGAPGPPGSPGPAGPTGKQGDRGEAGAQGPMGPSGPAGARG
IQGPQGPRGDKGEAGEPGERGLKGHRGFTGLQGLPGPPGPSGDQGASGPAGPSGPRGPP
GPVGPSGKDGANGIPGPIGPPGPRGRSGETGPAGPPGNPGPPGPPGPPGPGIDMSAFAGLG
PREKGPDPLQYMRA.

A long-term experimental research was performed to obtain a variant of a recombinant human type II collagen a1 chain, which was denoted as a1 (II) M6. Compared with the amino acid sequence of a1 (II), 9 amino acids were mutated to proline (Pro, abbreviated as P) in a1 (II) M6. That is, V at position 67, M at position 68, M at position 72, M at position 75, R at position 78, M at position 108, R at position 111, M at position 162, and R at position 165 in the amino acid sequence set forth in SEQ ID NO: 4 all were mutated to P, and the remaining amino acids in the amino acid sequence remained unchanged. A homology of a1 (II) M6 with a1 (II) was 99.2%.

The mutated amino acid sequence (a1 (II) M6) has a full length of 1,060 AA, and is set forth in SEQ ID NO: 5.

SEQ ID NO: 5:
QMAGGFDEKAGGAQLGPPQGPPGPPGPPGPPGPAGAPGPQGFQGNPGEPGEPGVS
GPPGPPGPPGPPGKPGDDGEAGKPGKAGERGPPGPQGARGFPGTPGLPGVKGHRGYPGL
DGAKGEAGAPGVKGESGSPGENGSPGPPGPPGLPGERGRTGPAGAAGARGNDGQPGPA
GPPGPVGPAGGPGFPGAPGAKGEAGPTGARGPEGAQGPRGEPGTPGSPGPAGASGNPGT
DGIPGAKGSAGAPGIAGAPGFPGPRGPPGPQGATGPLGPKGQTGEPGIAGFKGEQGPKGE
PGPAGPQGAPGPAGEEGKRGARGEPGGVGPIGPPGERGAPGNRGFPGQDGLAGPKGAP
GERGPSGLAGPKGANGDPGRPGEPGLPGARGLTGRPGDAGPQGKVGPSGAPGEDGRPG
PPGPQGARGQPGVMGFPGPKGANGEPGKAGEKGLPGAPGLRGLPGKDGETGAAGPPGP
AGPAGERGEQGAPGPSGFQGLPGPPGPPGEGGKPGDQGVPGEAGAPGLVGPRGERGFP
GERGSPGAQGLQGPRGLPGTPGTDGPKGASGPAGPPGAQGPPGLQGMPGERGAAGIAG
PKGDRGDVGEKGPEGAPGKDGGRGLTGPIGPPGPAGANGEKGEVGPPGPAGSAGARGA
PGERGETGPPGPAGFAGPPGADGQPGAKGEQGEAGQKGDAGAPGPQGPSGAPGPQGPT
GVTGPKGARGAQGPPGATGFPGAAGRVGPPGSNGNPGPPGPPGPSGKDGPKGARGDSG
PPGRAGEPGLQGPAGPPGEKGEPGDDGPSGAEGPPGPQGLAGQRGIVGLPGQRGERGFP
GLPGPSGEPGKQGAPGASGDRGPPGPVGPPGLTGPAGEPGREGSPGADGPPGRDGAAGV
KGDRGETGAVGAPGAPGPPGSPGPAGPTGKQGDRGEAGAQGPMGPSGPAGARGIQGPQ
GPRGDKGEAGEPGERGLKGHRGFTGLQGLPGPPGPSGDQGASGPAGPSGPRGPPGPVGP
SGKDGANGIPGPIGPPGPRGRSGETGPAGPPGNPGPPGPPGPPGPGIDMSAFAGLGPREKG
PDPLQYMRA.

Differences between the amino acid sequences of a1 (II) M6 and a1 (II) are shown by amino acids in bold and gray background in FIG. 2.

A DNA sequence of a gene (denoted as COL2AIM6) encoding the a1 (II) M6 set forth in SEQ ID NO: 5 is set forth in SEQ ID NO: 6.

SEQ ID NO: 6:
CAAATGGCTGGTGGATTCGATGAAAAGGCTGGTGGAGCCCAATTAGGTCCTCC
ACAAGGTCCTCCCGGTCCACCTGGTCCTCCCGGTCCTCCAGGTCCCGCCGGTGCTCC
TGGACCACAGGGTTTCCAAGGAAACCCCGGTGAACCAGGTGAGCCTGGTGTTTCAG
GTCCTCCCGGTCCTCCAGGACCACCTGGACCACCAGGAAAGCCTGGTGACGACGGA
GAAGCTGGTAAACCAGGAAAGGCAGGAGAGAGAGGTCCACCTGGACCTCAGGGTG
CCAGAGGTTTCCCAGGTACCCCTGGTCTTCCTGGTGTCAAGGGTCATAGAGGTTACC
CCGGTTTGGATGGTGCCAAGGGTGAAGCCGGTGCCCCTGGTGTTAAGGGTGAATCA
GGAAGTCCCGGTGAAAATGGAAGTCCCGGTCCACCCGGTCCACCTGGACTGCCAGG
TGAGAGAGGAAGAACCGGACCAGCTGGTGCTGCAGGTGCTAGAGGAAATGACGGA
CAGCCCGGACCAGCCGGACCTCCCGGTCCTGTTGGGCCCGCAGGTGGTCCTGGTTTC
CCtgGTGCTCCTGGAGCCAAAGGAGAAGCCGGACCCACCGGAGCCAGAGGTCCCGA
GGGAGCACAGGGACCTAGAGGAGAACCAGGTACACCAGGTAGTCCCGGTCCTGCTG
GTGCATCAGGAAATCCCGGAACTGACGGTATTCCAGGAGCAAAGGGATCTGCAGGA
GCACCAGGAATAGCTGGTGCTCCTGGATTTCCAGGTCCCAGAGGACCTCCCGGTCCT
CAAGGAGCAACAGGTCCTTTGGGACCAAAAGGTCAAACAGGAGAACCAGGTATTGC
TGGATTCAAAGGAGAGCAAGGTCCAAAGGGAGAGCCCGGTCCCGCAGGTCCCCAAG
GAGCCCCAGGACCAGCTGGTGAAGAAGGAAAAAGAGGAGCCAGAGGTGAACCTGG
AGGAGTAGGACCTATTGGTCCTCCTGGTGAGAGAGGTGCTCCCGGAAACAGAGGTT
TTCCTGGTCAAGATGGTCTGGCTGGACCTAAAGGTGCTCCAGGAGAGAGAGGACCT
TCAGGACTTGCTGGTCCAAAAGGTGCTAACGGAGATCCAGGAAGACCCGGTGAACC
TGGTCTGCCTGGAGCTAGAGGATTAACAGGAAGACCAGGTGACGCAGGTCCCCAGG
GTAAAGTGGGTCCCAGTGGTGCCCCAGGTGAAGATGGAAGACCTGGTCCTCCCGGA
CCCCAAGGTGCAAGAGGTCAGCCTGGAGTGATGGGATTTCCTGGACCCAAGGGTGC
TAACGGAGAACCTGGAAAAGCTGGTGAGAAAGGACTGCCCGGTGCCCCAGGTCTTA
GAGGTTTGCCAGGTAAAGATGGAGAAACAGGAGCCGCAGGACCACCCGGTCCAGCC
GGACCAGCAGGAGAGAGAGGTGAACAAGGAGCACCTGGTCCAAGTGGTTTTCAGGG
TCTTCCAGGTCCCCCTGGTCCACCAGGAGAGGGAGGTAAACCAGGTGACCAAGGTG
TCCCTGGAGAAGCAGGTGCACCCGGTCTTGTGGGTCCAAGAGGTGAAAGAGGATTC
CCTGGTGAGAGAGGATCTCCCGGAGCCCAGGGACTTCAAGGTCCTAGAGGTCTGCC
AGGTACCCCTGGTACAGACGGACCAAAGGGAGCATCAGGACCCGCTGGACCTCCCG
GAGCCCAAGGTCCTCCAGGTTTACAAGGTATGCCTGGTGAAAGAGGTGCTGCAGGT
ATAGCTGGACCAAAAGGAGACAGAGGTGACGTTGGTGAGAAGGGTCCCGAAGGAG
CCCCTGGAAAAGATGGTGGAAGAGGATTAACAGGTCCTATAGGACCACCCGGTCCA
GCCGGTGCTAATGGAGAAAAAGGAGAAGTAGGTCCTCCAGGTCCAGCAGGATCTGC
AGGTGCTAGAGGTGCCCCTGGAGAGAGAGGTGAAACAGGACCACCTGGTCCAGCTG
GTTTCGCTGGTCCCCCAGGAGCTGATGGACAGCCCGGTGCAAAAGGTGAACAAGGA
GAAGCCGGACAGAAGGGAGATGCTGGAGCCCCCGGTCCACAAGGTCCCTCAGGAGC
ACCAGGTCCTCAAGGTCCAACTGGTGTGACCGGGCCAAAGGGTGCAAGAGGAGCAC
AGGGACCTCCAGGAGCAACAGGTTTCCCAGGAGCTGCTGGTAGAGTCGGTCCACCC
GGATCTAATGGTAACCCCGGACCACCAGGACCACCTGGACCATCTGGAAAGGATGG
ACCCAAAGGAGCAAGAGGAGATTCAGGACCACCCGGAAGAGCAGGAGAACCTGGA
TTACAGGGTCCCGCCGGTCCACCAGGAGAGAAAGGAGAGCCCGGAGATGATGGTCC
CTCAGGTGCAGAGGGACCCCCAGGACCCCAAGGTCTGGCAGGTCAAAGAGGTATAG
TGGGTCTTCCAGGTCAAAGAGGTGAAAGAGGATTTCCAGGACTTCCAGGTCCTTCAG
GTGAACCCGGTAAACAGGGAGCCCCCGGAGCCTCAGGTGACAGAGGTCCTCCAGGA
CCAGTAGGACCCCCAGGTTTAACCGGACCAGCAGGTGAGCCAGGAAGAGAAGGTTC
TCCTGGAGCCGATGGACCTCCAGGAAGAGACGGTGCAGCTGGTGTTAAGGGTGACA
GAGGTGAAACTGGAGCCGTAGGAGCCCCAGGTGCCCCCGGACCACCCGGATCACCC
GGACCTGCAGGTCCTACTGGTAAACAAGGAGATAGAGGAGAAGCCGGTGCCCAGGG
TCCTATGGGTCCTTCTGGTCCTGCAGGAGCAAGAGGTATACAAGGTCCACAGGGTCC
CAGAGGTGACAAGGGTGAAGCAGGAGAACCCGGTGAGAGAGGTCTGAAGGGTCAT
AGAGGATTCACCGGGTTACAGGGTTTGCCAGGACCCCCTGGACCAAGTGGTGACCA
GGGTGCATCCGGTCCAGCAGGTCCTTCTGGACCAAGAGGTCCTCCCGGTCCAGTTGG
TCCATCAGGTAAAGACGGAGCCAACGGTATCCCAGGTCCCATCGGTCCTCCAGGTCC
TAGAGGAAGAAGTGGAGAGACTGGTCCTGCTGGACCTCCTGGAAACCCTGGTCCTC
CAGGACCTCCAGGTCCTCCAGGTCCCGGAATAGATATGTCCGCTTTCGCTGGATTGG
GACCAAGAGAGAAAGGTCCTGACCCTCTTCAATATATGAGAGCA.

Two termini of the DNA sequence encoding the a1 (1) M1 were modified as follows: a DNA sequence encoding a Strep-Tag II tag was added to the amino terminus and a DNA sequence encoding a 6×His Tag was added to the carboxyl terminus; and a1 (I) M1 protein with the tags was finally obtained by expression, which has 1,071 amino acids in total and is set forth in SEQ ID NO: 7.

SEQ ID NO: 7:
WSHPQFEKQLSYGYDEKSTGGISVPGPMGPSGPRGLPGPPGAPGPQGFQGPPGEPG
EPGASGPPGPPGPPGPPGKNGDDGEAGKPGRPGERGPPGPQGARGLPGTAGLPGMKGH
RGFSGLDGAKGDAGPAGPKGEPGSPGENGAPGQPGPPGLPGERGRPGAPGPAGARGND
GATGAAGPPGPTGPAGPPGFPGAVGAKGEAGPQGPRGSEGPQGVRGEPGPPGPAGAAG
PAGNPGADGQPGAKGANGAPGIAGAPGFPGARGPSGPQGPGGPPGPKGNSGEPGAPGS
KGDTGAKGEPGPVGVQGPPGPAGEEGKRGARGEPGPTGLPGPPGERGGPGSRGFPGAD
GVAGPKGPAGERGSPGPAGPKGSPGEAGRPGEAGLPGAKGLTGSPGSPGPDGKTGPPGP
AGQDGRPGPPGPPGARGQAGVMGFPGPKGAAGEPGKAGERGVPGPPGAVGPAGKDGE
AGAQGPPGPAGPAGERGEQGPAGSPGFQGLPGPAGPPGEAGKPGEQGVPGDLGAPGPS
GARGERGFPGERGVQGPPGPAGPRGANGAPGNDGAKGDAGAPGAPGSQGAPGLQGMP
GERGAAGLPGPKGDRGDAGPKGADGSPGKDGVRGLTGPIGPPGPAGAPGDKGESGPSG
PAGPTGARGAPGDRGEPGPPGPAGFAGPPGADGQPGAKGEPGDAGAKGDAGPPGPAGP
AGPPGPIGNVGAPGAKGARGSAGPPGATGFPGAAGRVGPPGPSGNAGPPGPPGPAGKEG
GKGPRGETGPAGRPGEVGPPGPPGPAGEKGSPGADGPAGAPGTPGPQGIAGQRGVVGLP
GQRGERGFPGLPGPSGEPGKQGPSGASGERGPPGPMGPPGLAGPPGESGREGAPGAEGS
PGRDGSPGAKGDRGETGPAGPPGAPGAPGAPGPVGPAGKSGDRGETGPAGPAGPVGPV
GARGPAGPQGPRGDKGETGEQGDRGIKGHRGFSGLQGPPGPPGSPGEQGPSGASGPAGP
RGPPGSAGAPGKDGLNGLPGPIGPPGPRGRTGDAGPVGPPGPPGPPGPPGPPSAGFDFSFL
PQPPQEKAHDGGRYYRAHHHHHH.

After optimization design, a DNA sequence of a gene (denoted as COLIAIMI) encoding the amino acid sequence (a1 (I) M1) set forth in SEQ ID NO: 7 is set forth in SEQ ID NO: 8.

SEQ ID NO: 8:
TGGTCTCATCCACAATTTGAAAAGCAACTTAGTTATGGATACGATGAAAAATCC
ACAGGTGGAATCAGTGTTCCTGGACCTATGGGTCCATCAGGTCCAAGAGGTTTACCA
GGACCTCCAGGTGCCCCAGGTCCCCAGGGATTTCAAGGTCCACCAGGAGAGCCTGG
TGAGCCAGGAGCTTCTGGTCCACCTGGTCCCCCTGGACCACCTGGTCCTCCAGGAAA
GAATGGAGATGATGGTGAAGCTGGAAAACCTGGAAGACCTGGAGAAAGAGGACCA
CCAGGACCCCAGGGTGCCAGAGGACTGCCAGGTACCGCAGGTCTGCCTGGAATGAA
AGGTCATAGAGGATTTTCAGGATTAGACGGTGCAAAGGGAGACGCTGGACCTGCAG
GACCAAAGGGTGAGCCAGGAAGTCCAGGAGAGAATGGTGCACCAGGACAGCCAGG
TCCACCTGGACTGCCCGGTGAAAGAGGTAGACCCGGAGCACCAGGACCAGCAGGTG
CAAGAGGAAATGATGGAGCTACAGGTGCTGCAGGACCCCCAGGTCCAACAGGACCA
GCCGGTCCTCCCGGTTTCCCAGGTGCCGTTGGAGCAAAAGGTGAAGCTGGTCCACA
GGGTCCAAGAGGTTCTGAAGGTCCACAGGGAGTTAGAGGAGAACCAGGACCCCCTG
GACCAGCTGGTGCAGCAGGACCAGCTGGTAACCCTGGTGCTGACGGTCAGCCAGGT
GCTAAGGGAGCAAATGGAGCACCAGGAATAGCTGGTGCCCCAGGATTTCCCGGTGC
TAGAGGTCCAAGTGGTCCACAAGGACCAGGAGGTCCACCCGGTCCCAAAGGAAACA
GTGGAGAACCAGGTGCACCCGGTTCAAAGGGAGATACAGGAGCTAAAGGAGAGCC
CGGTCCAGTGGGTGTTCAGGGACCACCCGGACCTGCTGGAGAGGAAGGTAAAAGAG
GTGCAAGAGGTGAGCCAGGACCAACAGGTCTGCCTGGTCCCCCTGGTGAAAGAGGT
GGTCCAGGTAGTAGAGGATTTCCAGGAGCTGATGGTGTTGCAGGACCAAAGGGACC
CGCAGGTGAGAGAGGATCACCCGGTCCAGCCGGACCAAAAGGATCACCAGGAGAA
GCTGGTAGACCAGGAGAAGCTGGTCTGCCAGGTGCTAAAGGATTGACAGGATCACC
CGGTTCACCTGGTCCTGATGGAAAGACAGGACCTCCAGGTCCCGCTGGTCAGGACG
GTAGACCAGGACCCCCAGGACCCCCAGGTGCAAGAGGTCAGGCAGGTGTAATGGGT
TTCCCCGGACCTAAAGGAGCAGCTGGAGAACCTGGTAAAGCTGGAGAGAGAGGAGT
GCCTGGACCCCCTGGAGCTGTTGGTCCAGCAGGAAAGGATGGTGAGGCAGGTGCAC
AAGGTCCACCTGGACCCGCTGGACCTGCAGGTGAGAGAGGAGAGCAAGGTCCCGCA
GGTTCTCCAGGTTTTCAGGGTTTGCCAGGTCCAGCCGGTCCTCCTGGAGAGGCAGGA
AAGCCAGGAGAACAAGGAGTTCCAGGAGACCTGGGTGCACCAGGACCCTCTGGTGC
AAGAGGAGAGAGAGGATTTCCTGGAGAAAGAGGTGTGCAGGGACCACCAGGTCCC
GCCGGTCCAAGAGGAGCAAATGGAGCCCCTGGAAATGACGGAGCTAAGGGTGACG
CTGGTGCACCAGGAGCACCAGGTTCTCAAGGTGCTCCCGGATTGCAGGGTATGCCTG
GAGAGAGAGGTGCAGCTGGACTGCCAGGTCCAAAAGGTGACAGAGGAGACGCCGG
TCCTAAGGGAGCTGACGGTTCTCCTGGAAAGGACGGTGTGAGAGGTTTGACAGGAC
CAATAGGTCCACCCGGTCCTGCTGGAGCCCCTGGAGACAAAGGTGAATCAGGTCCT
TCCGGTCCAGCCGGACCAACAGGAGCAAGAGGAGCACCTGGAGACAGAGGAGAGC
CAGGTCCTCCAGGACCTGCAGGTTTCGCTGGTCCTCCCGGAGCAGATGGACAGCCA
GGAGCTAAGGGAGAACCCGGTGACGCTGGTGCTAAGGGAGATGCAGGTCCACCAGG
TCCTGCTGGTCCTGCTGGACCTCCCGGACCAATAGGTAATGTTGGAGCACCCGGAGC
AAAAGGTGCCAGAGGTTCCGCAGGTCCTCCCGGAGCAACTGGTTTTCCAGGAGCTG
CCGGAAGAGTGGGTCCACCTGGTCCTTCTGGAAATGCAGGACCACCAGGTCCTCCTG
GTCCAGCCGGAAAGGAAGGTGGAAAGGGACCTAGAGGAGAAACAGGTCCCGCAGG
TAGACCCGGTGAGGTGGGTCCACCTGGTCCACCCGGTCCAGCTGGTGAGAAAGGAA
GTCCTGGAGCAGACGGACCAGCTGGTGCCCCTGGTACACCAGGACCCCAAGGAATA
GCTGGTCAAAGAGGTGTTGTTGGTTTACCAGGTCAGAGAGGAGAAAGAGGTTTTCC
AGGATTACCAGGTCCCTCAGGTGAGCCCGGAAAACAGGGTCCCTCAGGAGCAAGTG
GTGAAAGAGGACCACCAGGACCAATGGGACCTCCAGGATTAGCTGGTCCACCAGGA
GAATCAGGAAGAGAGGGTGCTCCTGGAGCAGAAGGTTCACCAGGAAGAGACGGTTC
ACCCGGAGCCAAGGGAGACAGAGGTGAAACAGGTCCCGCAGGTCCACCAGGAGCA
CCCGGAGCCCCTGGTGCTCCAGGACCTGTCGGACCAGCAGGAAAATCCGGTGACAG
AGGTGAGACTGGACCCGCAGGTCCTGCTGGTCCTGTTGGACCAGTGGGTGCAAGAG
GACCAGCAGGTCCACAAGGTCCAAGAGGTGACAAAGGTGAGACAGGTGAGCAGGG
TGACAGAGGAATTAAAGGTCACAGAGGATTTTCAGGACTGCAGGGACCACCCGGTC
CTCCCGGTTCCCCAGGAGAGCAAGGTCCATCCGGTGCATCCGGTCCAGCTGGACCCA
GAGGACCACCTGGTTCTGCTGGTGCACCAGGTAAAGATGGATTGAACGGTTTGCCTG
GTCCAATAGGACCTCCTGGTCCAAGAGGAAGAACTGGTGACGCCGGTCCCGTCGGA
CCACCCGGTCCACCAGGTCCCCCAGGTCCACCCGGACCACCATCCGCAGGATTTGAT
TTCTCATTCCTTCCTCAACCTCCTCAAGAGAAAGCACATGATGGAGGTAGATACTAT
AGAGCCCATCACCACCATCATCATTAA.

Two termini of the DNA sequence encoding the a1 (II) M6 were modified as follows: a DNA sequence encoding a Strep-Tag II tag was added to the amino terminus and a DNA sequence encoding a 6×His Tag was added to the carboxyl terminus; and a1 (II) M6 protein with the tags was finally obtained by expression, which has 1,076 amino acids in total and is set forth in SEQ ID NO: 9.

SEQ ID NO: 9:
EFWSHPQFEKQMAGGFDEKAGGAQLGPPQGPPGPPGPPGPPGPAGAPGPQGFQGN
PGEPGEPGVSGPPGPPGPPGPPGKPGDDGEAGKPGKAGERGPPGPQGARGFPGTPGLPG
VKGHRGYPGLDGAKGEAGAPGVKGESGSPGENGSPGPPGPPGLPGERGRTGPAGAAGA
RGNDGQPGPAGPPGPVGPAGGPGFPGAPGAKGEAGPTGARGPEGAQGPRGEPGTPGSP
GPAGASGNPGTDGIPGAKGSAGAPGIAGAPGFPGPRGPPGPQGATGPLGPKGQTGEPGIA
GFKGEQGPKGEPGPAGPQGAPGPAGEEGKRGARGEPGGVGPIGPPGERGAPGNRGFPG
QDGLAGPKGAPGERGPSGLAGPKGANGDPGRPGEPGLPGARGLTGRPGDAGPQGKVGP
SGAPGEDGRPGPPGPQGARGQPGVMGFPGPKGANGEPGKAGEKGLPGAPGLRGLPGKD
GETGAAGPPGPAGPAGERGEQGAPGPSGFQGLPGPPGPPGEGGKPGDQGVPGEAGAPG
LVGPRGERGFPGERGSPGAQGLQGPRGLPGTPGTDGPKGASGPAGPPGAQGPPGLQGM
PGERGAAGIAGPKGDRGDVGEKGPEGAPGKDGGRGLTGPIGPPGPAGANGEKGEVGPP
GPAGSAGARGAPGERGETGPPGPAGFAGPPGADGQPGAKGEQGEAGQKGDAGAPGPQ
GPSGAPGPQGPTGVTGPKGARGAQGPPGATGFPGAAGRVGPPGSNGNPGPPGPPGPSGK
DGPKGARGDSGPPGRAGEPGLQGPAGPPGEKGEPGDDGPSGAEGPPGPQGLAGQRGIV
GLPGQRGERGFPGLPGPSGEPGKQGAPGASGDRGPPGPVGPPGLTGPAGEPGREGSPGA
DGPPGRDGAAGVKGDRGETGAVGAPGAPGPPGSPGPAGPTGKQGDRGEAGAQGPMGP
SGPAGARGIQGPQGPRGDKGEAGEPGERGLKGHRGFTGLQGLPGPPGPSGDQGASGPA
GPSGPRGPPGPVGPSGKDGANGIPGPIGPPGPRGRSGETGPAGPPGNPGPPGPPGPPGPGI
DMSAFAGLGPREKGPDPLQYMRAHHHHHH.

After optimization design, a DNA sequence of a gene (denoted as COL2A1M6) encoding the amino acid sequence (a1 (II) M6) set forth in SEQ ID NO: 9 is set forth in SEQ ID NO: 10.

SEQ ID NO: 10:
GAATTCTGGAGTCATCCTCAATTCGAAAAACAAATGGCTGGTGGATTCGATGAA
AAGGCTGGTGGAGCCCAATTAGGTCCTCCACAAGGTCCTCCCGGTCCACCTGGTCCT
CCCGGTCCTCCAGGTCCCGCCGGTGCTCCTGGACCACAGGGTTTCCAAGGAAACCCC
GGTGAACCAGGTGAGCCTGGTGTTTCAGGTCCTCCCGGTCCTCCAGGACCACCTGGA
CCACCAGGAAAGCCTGGTGACGACGGAGAAGCTGGTAAACCAGGAAAGGCAGGAG
AGAGAGGTCCACCTGGACCTCAGGGTGCCAGAGGTTTCCCAGGTACCCCTGGTCTTC
CTGGTGTCAAGGGTCATAGAGGTTACCCCGGTTTGGATGGTGCCAAGGGTGAAGCC
GGTGCCCCTGGTGTTAAGGGTGAATCAGGAAGTCCCGGTGAAAATGGAAGTCCCGG
TCCACCCGGTCCACCTGGACTGCCAGGTGAGAGAGGAAGAACCGGACCAGCTGGTG
CTGCAGGTGCTAGAGGAAATGACGGACAGCCCGGACCAGCCGGACCTCCCGGTCCT
GTTGGGCCCGCAGGTGGTCCTGGTTTCCCtgGTGCTCCTGGAGCCAAAGGAGAAGCC
GGACCCACCGGAGCCAGAGGTCCCGAGGGAGCACAGGGACCTAGAGGAGAACCAG
GTACACCAGGTAGTCCCGGTCCTGCTGGTGCATCAGGAAATCCCGGAACTGACGGT
ATTCCAGGAGCAAAGGGATCTGCAGGAGCACCAGGAATAGCTGGTGCTCCTGGATT
TCCAGGTCCCAGAGGACCTCCCGGTCCTCAAGGAGCAACAGGTCCTTTGGGACCAA
AAGGTCAAACAGGAGAACCAGGTATTGCTGGATTCAAAGGAGAGCAAGGTCCAAA
GGGAGAGCCCGGTCCCGCAGGTCCCCAAGGAGCCCCAGGACCAGCTGGTGAAGAA
GGAAAAAGAGGAGCCAGAGGTGAACCTGGAGGAGTAGGACCTATTGGTCCTCCTGG
TGAGAGAGGTGCTCCCGGAAACAGAGGTTTTCCTGGTCAAGATGGTCTGGCTGGAC
CTAAAGGTGCTCCAGGAGAGAGAGGACCTTCAGGACTTGCTGGTCCAAAAGGTGCT
AACGGAGATCCAGGAAGACCCGGTGAACCTGGTCTGCCTGGAGCTAGAGGATTAAC
AGGAAGACCAGGTGACGCAGGTCCCCAGGGTAAAGTGGGTCCCAGTGGTGCCCCAG
GTGAAGATGGAAGACCTGGTCCTCCCGGACCCCAAGGTGCAAGAGGTCAGCCTGGA
GTGATGGGATTTCCTGGACCCAAGGGTGCTAACGGAGAACCTGGAAAAGCTGGTGA
GAAAGGACTGCCCGGTGCCCCAGGTCTTAGAGGTTTGCCAGGTAAAGATGGAGAAA
CAGGAGCCGCAGGACCACCCGGTCCAGCCGGACCAGCAGGAGAGAGAGGTGAACA
AGGAGCACCTGGTCCAAGTGGTTTTCAGGGTCTTCCAGGTCCCCCTGGTCCACCAGG
AGAGGGAGGTAAACCAGGTGACCAAGGTGTCCCTGGAGAAGCAGGTGCACCCGGTC
TTGTGGGTCCAAGAGGTGAAAGAGGATTCCCTGGTGAGAGAGGATCTCCCGGAGCC
CAGGGACTTCAAGGTCCTAGAGGTCTGCCAGGTACCCCTGGTACAGACGGACCAAA
GGGAGCATCAGGACCCGCTGGACCTCCCGGAGCCCAAGGTCCTCCAGGTTTACAAG
GTATGCCTGGTGAAAGAGGTGCTGCAGGTATAGCTGGACCAAAAGGAGACAGAGGT
GACGTTGGTGAGAAGGGTCCCGAAGGAGCCCCTGGAAAAGATGGTGGAAGAGGATT
AACAGGTCCTATAGGACCACCCGGTCCAGCCGGTGCTAATGGAGAAAAAGGAGAAG
TAGGTCCTCCAGGTCCAGCAGGATCTGCAGGTGCTAGAGGTGCCCCTGGAGAGAGA
GGTGAAACAGGACCACCTGGTCCAGCTGGTTTCGCTGGTCCCCCAGGAGCTGATGG
ACAGCCCGGTGCAAAAGGTGAACAAGGAGAAGCCGGACAGAAGGGAGATGCTGGA
GCCCCCGGTCCACAAGGTCCCTCAGGAGCACCAGGTCCTCAAGGTCCAACTGGTGT
GACCGGGCCAAAGGGTGCAAGAGGAGCACAGGGACCTCCAGGAGCAACAGGTTTC
CCAGGAGCTGCTGGTAGAGTCGGTCCACCCGGATCTAATGGTAACCCCGGACCACC
AGGACCACCTGGACCATCTGGAAAGGATGGACCCAAAGGAGCAAGAGGAGATTCA
GGACCACCCGGAAGAGCAGGAGAACCTGGATTACAGGGTCCCGCCGGTCCACCAGG
AGAGAAAGGAGAGCCCGGAGATGATGGTCCCTCAGGTGCAGAGGGACCCCCAGGA
CCCCAAGGTCTGGCAGGTCAAAGAGGTATAGTGGGTCTTCCAGGTCAAAGAGGTGA
AAGAGGATTTCCAGGACTTCCAGGTCCTTCAGGTGAACCCGGTAAACAGGGAGCCC
CCGGAGCCTCAGGTGACAGAGGTCCTCCAGGACCAGTAGGACCCCCAGGTTTAACC
GGACCAGCAGGTGAGCCAGGAAGAGAAGGTTCTCCTGGAGCCGATGGACCTCCAGG
AAGAGACGGTGCAGCTGGTGTTAAGGGTGACAGAGGTGAAACTGGAGCCGTAGGA
GCCCCAGGTGCCCCCGGACCACCCGGATCACCCGGACCTGCAGGTCCTACTGGTAA
ACAAGGAGATAGAGGAGAAGCCGGTGCCCAGGGTCCTATGGGTCCTTCTGGTCCTG
CAGGAGCAAGAGGTATACAAGGTCCACAGGGTCCCAGAGGTGACAAGGGTGAAGC
AGGAGAACCCGGTGAGAGAGGTCTGAAGGGTCATAGAGGATTCACCGGGTTACAGG
GTTTGCCAGGACCCCCTGGACCAAGTGGTGACCAGGGTGCATCCGGTCCAGCAGGT
CCTTCTGGACCAAGAGGTCCTCCCGGTCCAGTTGGTCCATCAGGTAAAGACGGAGCC
AACGGTATCCCAGGTCCCATCGGTCCTCCAGGTCCTAGAGGAAGAAGTGGAGAGAC
TGGTCCTGCTGGACCTCCTGGAAACCCTGGTCCTCCAGGACCTCCAGGTCCTCCAGG
TCCCGGAATAGATATGTCCGCTTTCGCTGGATTGGGACCAAGAGAGAAAGGTCCTG
ACCCTCTTCAATATATGAGAGCACACCATCACCATCATCACTAA.

The synthesis of DNA sequences was entrusted to Nanjing Genscript Biotechnology Co., Ltd. to synthesize DNA fragments of the genes set forth in SEQ ID NO: 8 and SEQ ID NO: 10.

### Example 2 Construction of Recombinant Expression Vectors and Screening of Strains

### (1) Construction of Recombinant Expression Vectors

Synthesized gene fragments set forth in SEQ ID NO: 8 and SEQ ID NO: 10 were recombined into a pPIC9K empty vector (purchased from Thermo Fisher Scientific) with a target fragment being accurately inserted into a secretion signal a factor-containing reading frame of a secretory vector to obtain a recombinant expression vector plasmid pPIC9K-COL2AIM6 expressing a1 (II) M6 and a recombinant expression vector plasmid pPIC9K-COLIAIMI expressing a1 (I) M1.

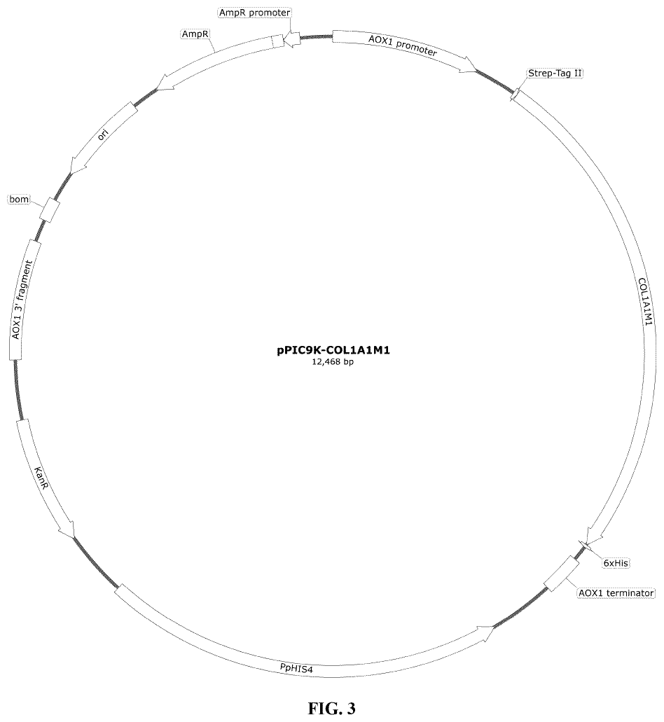

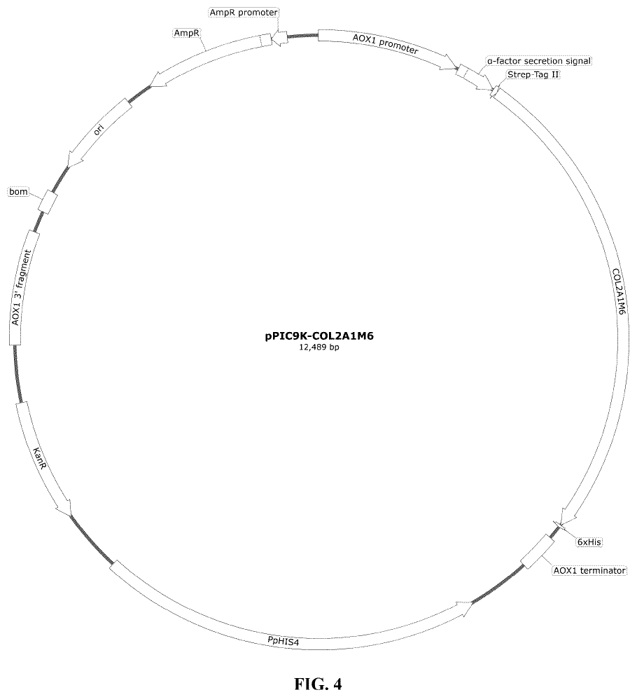

The plasmids pPIC9K-COL2A1M6 and pPIC9K-COLIAIMI were transformed into competent Escherichia coli DH5a (purchased from the Sangon Biotech (Shanghai) Co., Ltd.), positive clones were screened on an LB resistant plate with ampicillinum, and the recombinant plasmids were extracted for sequencing (which was entrusted to the Sangon Biotech (Shanghai) Co., Ltd.). Sequencing results showed that the recombinant plasmids were correct. Maps of the plasmids pPIC9K-COLIAIMI and pPIC9K-COL2AIM6 are shown in FIG. 3 and FIG. 4, respectively.

### (2) Screening of Strains

10 μg of the recombinant expression vector plasmids was digested overnight at 37° C. with SacI (SacI was purchased from the Dalian TaKaRa, and specific operations were performed according to instructions of a kit) for linearization, and then a PCR product purification kit (purchased from the Sangon Biotech (Shanghai) Co., Ltd.) was used to recover a linearized plasmid, with a volume controlled at about 10 μL.

The linearized plasmid was electrotransformed into a host Pichia pastoris SMD1168 (purchased from Thermo Fisher Scientific) competent cell, an electrotransformed Pichia pastoris solution was coated on MD plates with 100 μL to 200 μL for each plate, and the plates were allowed to stand at room temperature for 10 min and invertedly incubated at 30° C. for 2 d to 5 d until single colonies (positive transformants) appeared.

2 mL of sterile double-distilled water was added to a surface of each MD plate, then His⁺ transformants on the surface of the plate were gently scraped off with a sterile triangular spreader, transferred to a 50 mL centrifuge tube, and diluted with sterile double-distilled water to obtain a suspension, 10⁵ cells were coated on a YPD plate including 0.5 mg/mL G418, and the plate was inverted and incubated at 30° C. for 3 d to 4 d until single colonies appeared. Colonies were picked from the YPD plate and added to a first sterile 96-well plate (200 μL of YPD/well), and a resulting mixture in each well was thoroughly mixed and incubated at 30° C. for 48 h to obtain a first cultivation system. The first cultivation system in each well of the first sterile 96-well plate was thoroughly mixed, and 10 μL of the first cultivation system was taken, added to a second sterile 96-well plate, and then incubated at 30° C. for 24 h to obtain a second cultivation system. The second cultivation system in each well of the second sterile 96-well plate was thoroughly mixed, and 10 μL of the second cultivation system was taken, added to a third sterile 96-well plate, and then incubated at 30° C. for 24 h to obtain a third cultivation system. 1 μL of the third cultivation system in the third sterile 96-well plate was taken, spotted on YPD plates with 1.0 mg/mL and 4 mg/mL G418, and further incubated at 30° C. for 96 h to 120 h. If Pichia pastoris transformants could grow on a plate with a high G418 concentration (4 mg/mL), it indicated that the transformants carried a plurality of copies of a target gene, that is, a plurality of recombinant fragments had entered Pichia pastoris and had been integrated into a chromosome of Pichia pastoris through homologous recombination. After the screening in this step, a recombinant engineered Pichia pastoris strain with a high copy number and a high expression level was obtained.

The two engineered strain samples respectively carrying pPIC9K-COLIAIMI and pPIC9K-COL2AIM6 constructed were deposited in the China General Microbiological Culture Collection Center (CGMCC).

The engineered strain carrying the recombinant expression vector pPIC9K-COLIAIMI could express the recombinant collagen a1 (I) M1, and was deposited in the China General Microbiological Culture Collection Center (CGMCC) located at NO. 1, West Beichen Road, Chaoyang District, Beijing, China on Mar. 11, 2021, with an accession number of CGMCC NO. 21891 and a taxonomic name of Pichia pastoris.

The engineered strain carrying the recombinant expression vector pPIC9K-COL2AIM6 could express the recombinant collagen a1 (II) M6, and was deposited in the China General Microbiological Culture Collection Center (CGMCC) located at NO. 1, West Beichen Road, Chaoyang District, Beijing, China on Mar. 11, 2021, with an accession number of CGMCC NO. 21892 and a taxonomic name of Pichia pastoris.

### Example 3 Inducible Expression and Identification of Recombinant Collagen

The recombinant engineered strains expressing a1 (I) M1 and a1 (II) M6 obtained in Example 2 were taken, and an engineered Pichia pastoris strain expressing a full-length type I collagen a1 chain and an engineered Pichia pastoris strain expressing a full-length type II collagen a1 chain in the known patents were taken as controls. The two control engineered strains were the previous research results of the team of the inventors, and full-length collagen a1 chains expressed by the two control engineered strains also had a Strep-Tag II at an amino terminus and a 6×His Tag at a carboxyl terminus. The two control engineered strains were from the application No. 201911135958.0 (title: Yeast Recombinant Human Type I Collagen a1 Chain and Synthesis Method and Use thereof, an engineered Pichia pastoris strain expressing a full-length a1 (I) chain in this patent was deposited in the China General Microbiological Culture Collection Center (CGMCC), with an accession number of CGMCC NO. 17150) and the application No. 201911088025.0 (title: Method for Producing Recombinant Human Type II Collagen Single-Chain with Pichia pastoris, an engineered Pichia pastoris strain expressing a full-length a1 (II) chain in this patent was deposited in the China General Microbiological Culture Collection Center (CGMCC), with an accession number of CGMCC NO. 17149). The four engineered strains each were placed in a 100 mL erlenmeyer flask filled with 10 mL of a BMGY medium, and cultivated at 28° C. to 30° C. and 220 rpm until OD₆₀₀ was 2 to 6 (16 h to 18 h) to obtain a cultivation system. The cultivation system was centrifuged at 1,500 g to 3,000 g for 5 min at room temperature to obtain a precipitate, the precipitate was resuspended in a BMMY medium to obtain a suspension with OD₆₀₀ of about 2, and then the suspension was cultivated on a shaker at 28° C.′ to 30° C. and 220 rpm for 3 d, during which 100% methanol was added to the medium every 24 h to make a final concentration of methanol in the medium was 1.0%. After the induction with methanol was performed for 16 h or more, 1 mL of a strain solution sample was collected, placed in a 1.5 mL EP tube, and centrifuged at 12,000 g and 4° C. for 5 min to obtain an expression supernatant, and the expression supernatant was collected and stored at −80° C. for later use.

A 5× loading buffer (250 mM Tris-HCl, pH 6.8, 10% SDS, 0.5% bromophenol blue, 50% glycerin, and 5% β-mercaptoethanol) was added to the collected expression supernatant, and a resulting mixed solution was heated in a metal bath at 100° C. for 10 min and then tested by SDS-PAGE. Because the expressed target protein included a Srtep-Tag II at an amino terminus and a 6×His Tag at a carboxyl terminus, WB could be performed with an anti-Srtep-Tag II antibody and an anti-6×His Tag antibody (purchased from Nanjing Genscript Biotechnology Co., Ltd.) (specific operations could refer to instructions).

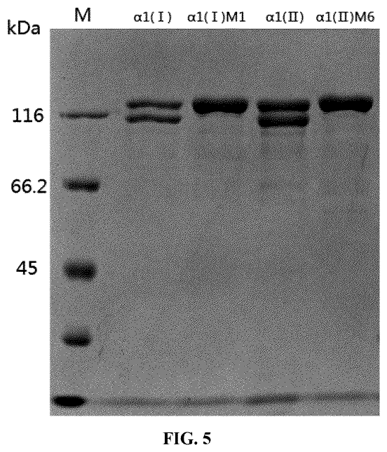

SDS-PAGE results of expression supernatants are shown in FIG. 5. a1 (I) M1, a1 (II) M6, a1 (1), and a1 (II) could be efficiently produced by secretory expression in an extracellular expression supernatant after 24 h of inducible expression. a1 (I) and a1 (II) each have a clear main degradation band (<116 kDa) below an expected target band (>116 kDa), while a1 (I) M1 and a1 (II) M6 of the present disclosure each merely have an expected target band (>116 kDa).

The measurement was performed with the Image Lab software (Bio-Rad Gel Doc XR+Imager), and measurement results were as follows.

(1) An apparent molecular weight (116.3 kDa) of a target band of a1 (I) M1 was substantially consistent with an apparent molecular weight (116.4 kDa) of a target band of a1 (I), and an apparent molecular weight (118.2 kDa) of a target band of a1 (II) M6 was substantially consistent with an apparent molecular weight (118.1 kDa) of a target band of a1 (II). Apparent molecular weights of the target band of a1 (I) M1 and the target band of a1 (II) M6 were significantly larger than an apparent molecular weight of the main degradation band (104.5 kDa) of a1 (I) and an apparent molecular weight of the main degradation band (106.9 kDa) of a1 (II).

(2) A ratio of a target band to a main degradation band in an electrophoresis result of a1 (I) was 51.5%: 48.3%, and a ratio of a target band to a main degradation band in an electrophoresis result of a1 (II) was 52.1%: 47.8%. The two ratios were substantially the same.

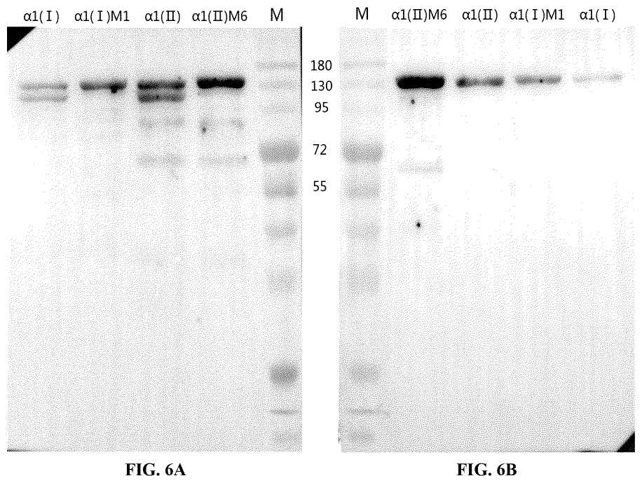

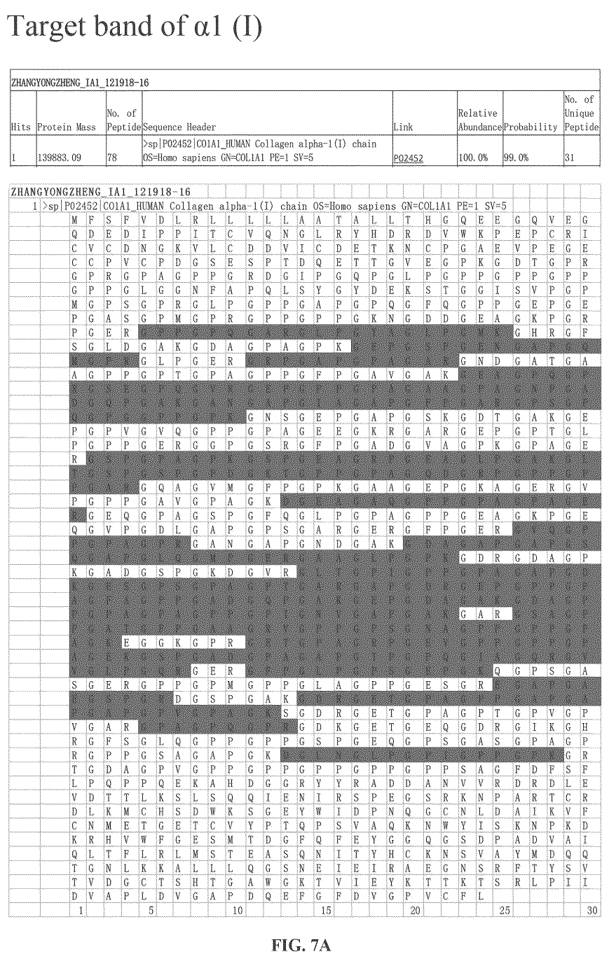

It can be seen from enhanced chemiluminescence (ECL) color development results in FIGS. 6A-6B (a fully automatic chemiluminescence image analysis system Tanon 5200 integrates protein molecular weight markers into images) that the Srtep-Tag II tag at the amino terminus and the 6×His Tag at the carboxyl terminus both could be detected in the recombinant collagen a1 (I) M1 and a1 (II) M6, and target bands have the same apparent molecular weights as in SDS-PAGE, indicating that full-length sequences of the recombinant collagen a1 (I) M1 and a1 (II) M6 are successfully and efficiently produced by secretory expression and the expression of target bands is as expected. Although sequences of target bands of a1 (I) and a1 (II) collagens are full-length and complete, main degradation bands of a1 (I) and a1 (II) lack the amino-terminal sequence, and only the carboxyl-terminal 6×His Tag can be detected.

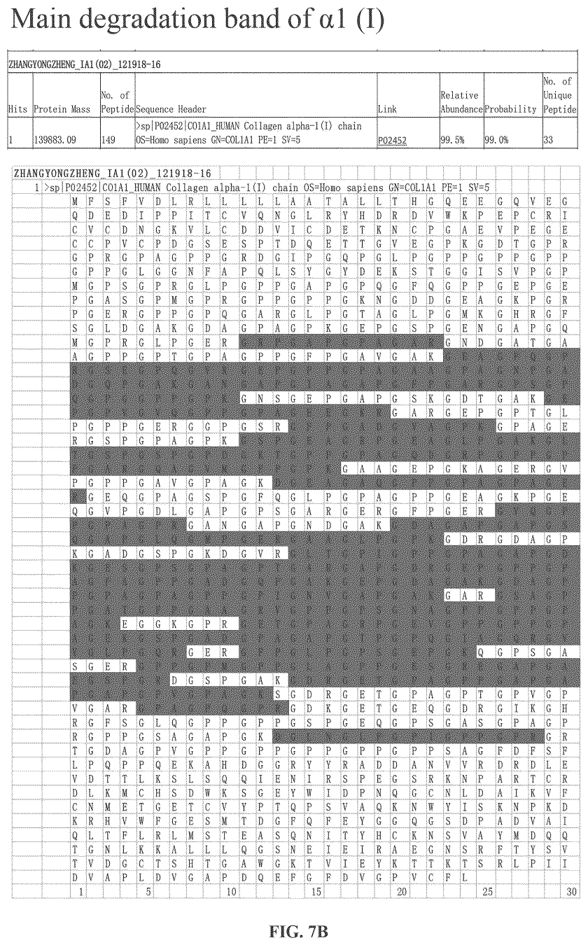

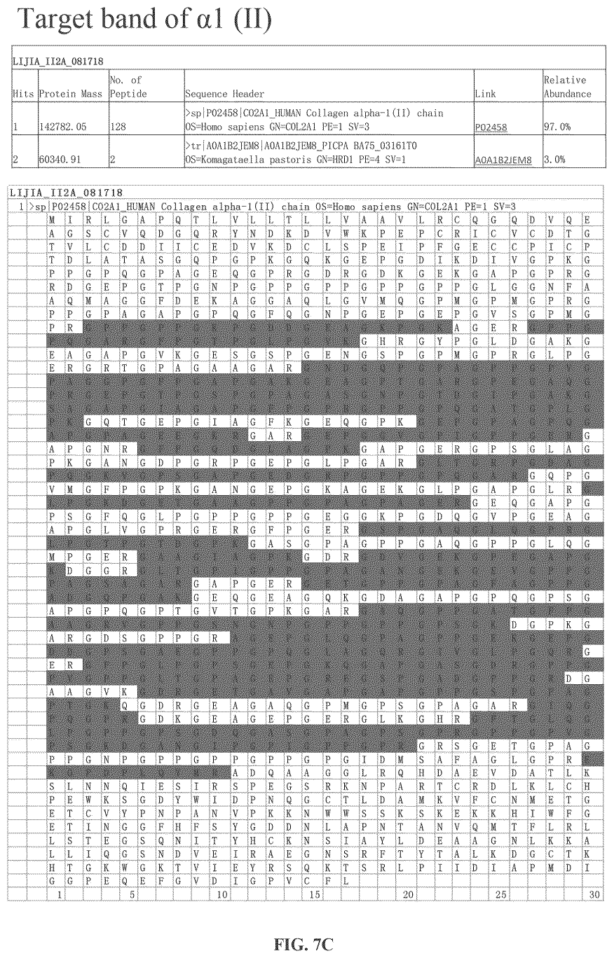

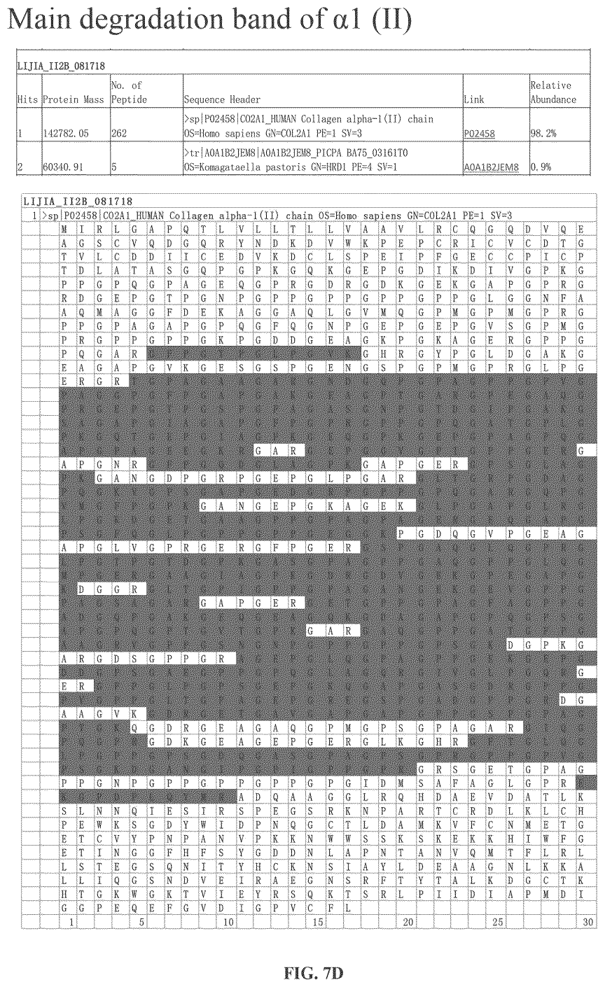

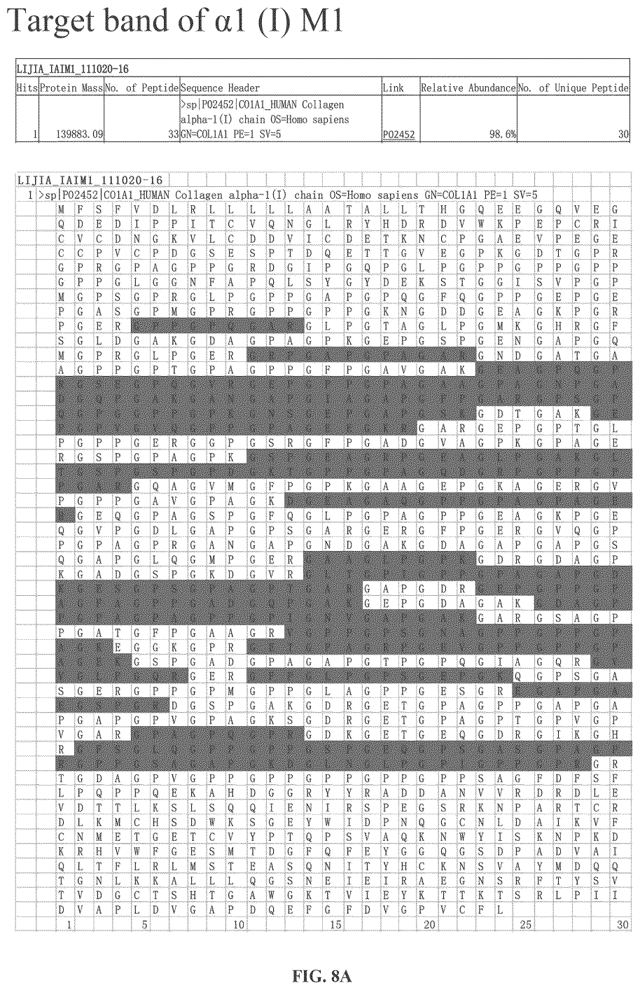

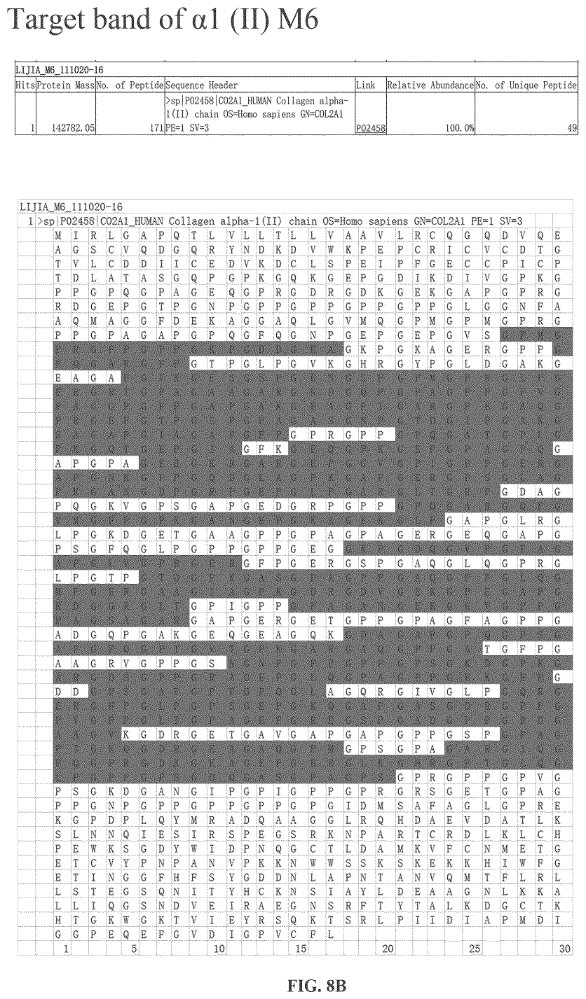

Target bands of a1 (I) M1 and a1 (II) M6 on an SDS-PAGE pattern, target bands of a1 (I) and a1 (II) on an SDS-PAGE pattern, and main degradation bands were cut off, subjected to enzymolysis with trypsin, and then tested by Nano-HPLC-MS/MS (which was entrusted to Suzhou ProtTech Inc.). Detected peptides were subjected to sequence alignment (Uniprot database), and data alignment results and alignment coverage maps of identified peptides with native sequences (parts with gray background, indicating parts of a peptide identified by mass spectrometry in a band that were identical to a native sequence) are shown in FIG. 7A-FIG. 7D and FIG. 8A-FIG. 8B. It could be found that:

(1) Peptides detected after enzymolysis of target bands of a1 (I) M1 and a1 (I) and a main degradation band of a1 (I) all were sequences on a type I collagen a1 chain.

(2) Peptides detected after enzymolysis of target bands of a1 (II) M6 and a1 (II) and a main degradation band of a1 (II) all were sequences on a type II collagen a1 chain.

The above results showed that a1 (I) M1 and a1 (II) M6 were expressed as successfully as a1 (I) and a1 (II), and were recombinant collagens of a human type I collagen a1 chain and a human type II collagen a1 chain, respectively, but a1 (I) and a1 (II) were degraded during expression, and main degradation bands of a1 (I) and a1 (II) also were collagens of respective types.

### Example 4 High-Density Fermentation and Purification

(1) High-Density Fermentation with Genetically Engineered Strains

The recombinant collagen a1 (I) M1 and a1 (II) M6 were produced by expression on a large scale to obtain fermentation broths containing the recombinant collagen a1 (I) M1 and a1 (II) M6, respectively.

A seed medium YPG (yeast powder: 10 g/L, yeast peptone: 20 g/L, and anhydrous glycerin: 10 g/L); a fermentation medium (NH₄H₂PO₄: 190.4 g/L, KH₂PO₄: 10.06 g/L, CaSO₄·2H₂O: 1.18 g/L, K₂SO₄: 18.2 g/L, MgSO₄·7H₂O: 14.9 g/L, and glycerin: 40 g/L); a feed medium (including 50% W/V glycerin and 12 mL of PTM1 trace salts per liter); and an induction medium (including 100% methanol and 12 mL of PTM1 trace salts per liter) were adopted. The PTM1 was filtered through a 0.22 μm filter membrane for sterilization and stored at 4° C. The fermentation medium was sterilized at a high temperature and cooled to room temperature, then PTM1 was added to obtain a PTM1-containing fermentation medium, and the pH of the PTM1-containing fermentation medium was adjusted with ammonia water to 5.0.

Batch-cultivation and inducible expression of the constructed engineered strains were performed as follows.

The fed-batch cultivation was performed at 30° C.

The engineered strain was inoculated into a 1 L shake flask with the seed medium YPG, and cultivated at 220 rpm and 30° C. for 18 h to 20 h until OD₆₀₀ was 2 to 10. 5 L fermentation tanks (Baoxing Biological Equipment Co., Ltd.) were used to be filled with 2 L of the fermentation medium containing 2% glycerin, and were sterilized separately. Before inoculation, a rotational speed was adjusted to 300 rpm, a ventilation rate was adjusted to 4 L/min, a temperature was adjusted to 30° C., and a pH was adjusted with an alkaline solution prepared from concentrated ammonia water to 4.5. 0.9 mL of PTM1 was added to the fermentation tank, and then 200 mL of a seed solution prepared was inoculated in the fermentation tank (flame ring inoculation). Then a dissolved oxygen electrode was clicked for hundred calibration, and fermentation was started. When the dissolved oxygen level fell to 30% for the first time, the dissolved oxygen level was kept at 30% by the dissolved oxygen/rotational speed cascade function. After glycerin was exhausted and the dissolved oxygen level rebounded to greater than 70% (OD₆₀₀ about 20), the dissolved oxygen/rotational speed cascade function was canceled, the stirring speed was increased to 650 rpm, and 80 mL of 30% glycerin was supplemented through linkage feeding. After the glycerin supplementation was completed and the dissolved oxygen level rebounded to 70% or more, the pH was set to 4 and the temperature was set to 29° C. to allow induced cultivation with a mixed carbon source of methanol and glycerin (methanol: 50% glycerin=7:3). 5 mL of the mixed carbon source was manually added. After the dissolved oxygen level rebounded to 70% or more, the feeding rate was set to 8 mL/h, then increased to 10 mL/h one hour later, and then further increased to 20 mL/h one hour later. When the dissolved oxygen level was lower than 30%, the feeding was stopped. After the dissolved oxygen level rebounded to 30%, linkage feeding was started. When a protein concentration measured by a UV method did not increase significantly or decreased after 40 h to 60 h of induction, a resulting fermentation broth was discharged. UV protein quantification formula: C (mg/mL)=0.144* (A215-A225), A215<1.5. An engineered Pichia pastoris strain expressing a full-length a1 (I) chain (which was deposited in the China General Microbiological Culture Collection Center (CGMCC), with an accession number of CGMCC NO. 17150) and an engineered Pichia pastoris strain expressing a full-length a1 (II) chain (which was deposited in the China General Microbiological Culture Collection Center (CGMCC), with an accession number of CGMCC NO. 17149) each were used for high-density fermentation.

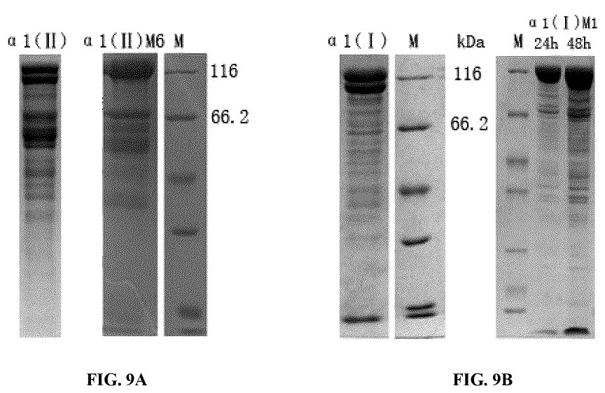

Results were shown in Table 1. After 48 h of induction, there was no significant difference between a1 (I) and a1 (I) M1 and between a1 (II) M6 and a1 (II) in terms of a strain concentration (OD₆₀₀), a strain wet weight, and a UV-quantified protein concentration expressed in a fermentation broth. Fermentation supernatants were collected and tested by SDS-PAGE, and test results are shown in FIGS. 9A-9B. Under high-density fermentation conditions, main degradation bands of a1 (I) and a1 (II) were extremely obvious, which had no difference from the inducible expression in shake flasks. Main products of a1 (I) M1 and a1 (II) M6 were still target bands, and no main degradation band occurred, indicating that effects of a1 (I) M1 and a1 (II) M6 for eliminating main degradation bands (main degradation products) could still be effectively maintained under the high-density fermentation conditions.

TABLE 1
Strain concentrations, strain wet weights, and protein expression
levels (UV quantification) in fermentation pilot experiments

| Type | OD₆₀₀ | Strain wet weight (g/L) | Protein (UV, g/L) |
|---|---|---|---|
| α1(II) | 189.0 | 260.0 | 17.8 |
| α1(II)M6 | 198.0 | 265.0 | 18.7 |
| α1(I) | 215.0 | 310.0 | 18.1 |
| α1(I)M1 | 201.0 | 301.0 | 17.3 |

### (2) Collagen Purification

Buffer A: 20 mM KH₂PO₄, pH 4.0; and
buffer B: 20 mM KH₂PO₄, 0.5 M NaCl, pH 4.0.

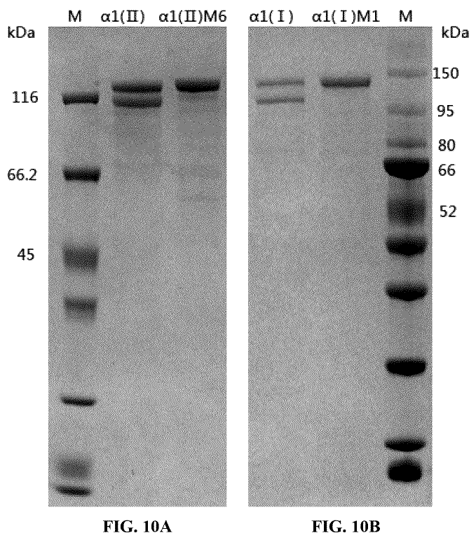

A fermentation broth was collected and centrifuged at 2,000 g and 4° C. for 30 min to obtain a strain precipitate and a fermentation supernatant. A cation exchange medium (a chromatography packing was UniGel-80sp produced by Suzhou Nanomicro Technology Co., Ltd., the chromatography packing was loaded on a GCC-50-400 chromatography column produced by the Lisure Science, and a GE AKTA Pure Protein Separation Chromatography Purification System was adopted) was equilibrated with the buffer A until an A215 absorbance value and a conductivity value remained unchanged, and then a sample was loaded at a flow rate of 40 us/cm and a volume of 0.5 L/time. The UV A215 absorbance was detected, and when it increased, the collection of an effluent was started. When the sample loading was completed, the collection of an effluent was stopped, and then the cation exchange medium was equilibrated with the buffer A. When the A215 absorbance decreased, the collection of an effluent was started until the UV absorbance and conductivity dropped to minimum values and no longer changed. An eluate was collected, tested to determine a composition, and then subjected to dialysis (with ultrapure water as a dialysis solution), concentration, and lyophilization to obtain a lyophilized collagen sponge, and the lyophilized collagen sponge was collected, dissolved in ultrapure water, and subjected to SDS-PAGE. Results are shown in FIGS. 10A-10B. After one-step ion exchange purification of a1 (I) M1 and a1 (II) M6, most of heteroproteins and small degradation bands were removed, and single target proteins with high purities were obtained (according to measurement results of the Image Lab software, a purity of a1 (I) M1 was 90.1% and a purity of a1 (II) M6 was 88.3%). However, after the same purification step of a1 (1) and a1 (II), a main degradation band still appeared and could not be eliminated, and a main degradation product had a similar size and very similar properties to a target product, such that the main degradation product and the target product could hardly be separated through one-step purification. According to the content in the applications Ser. No. 20/191,1135958.0 and No. 201911088025.0, in order to obtain single target full-length a1 (I) and a1 (II) chain products, tandem affinity purification needed to be performed with Ni-NTA and Strep-Tactin affinity chromatography media based on the fact that a full-length a1 (I) chain and a full-length a1 (II) chain had a Srtep-Tag II at an amino terminus and a 6×His Tag at a carboxyl terminus. In this case, a main degradation product presented as a main degradation band during SDS-PAGE was discarded, which caused a waste of a biosynthetic resource of a strain, increased purification steps, and reduced a yield of a target product.

### Example 5 Detection of Recombinant Collagens

### (1) FT-IR Spectroscopy

A trace amount of each of the purified and lyophilized a1 (I) M1, a1 (II) M6, a1 (I), and a1 (II) collagen samples was taken for test, mixed with potassium bromide (KBr), ground into a powder, then pressed into a tablet, and scanned in a range of 4,000 cm⁻¹ to 400 cm⁻¹ at room temperature (Thermo Scientific, Nicolet™ iS™ 10) FT-IR Spectrometer). The method and result analysis could be found in the literature (Jeong, H., J. Venkatesan and S. Kim, Isolation and characterization of collagen from marine fish (Thunnus obesus). Biotechnology and Bioprocess Engineering, 2013. 18 (6): p. 1185-1191.).

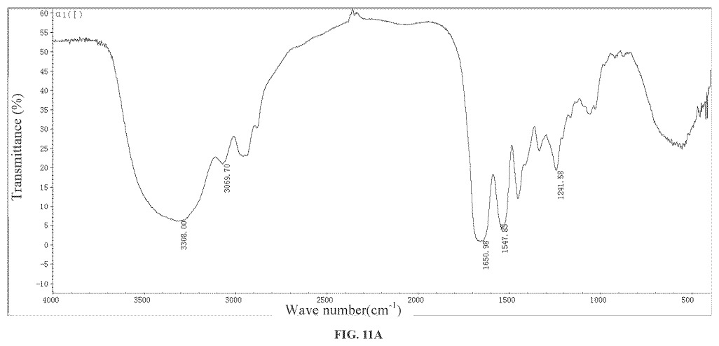

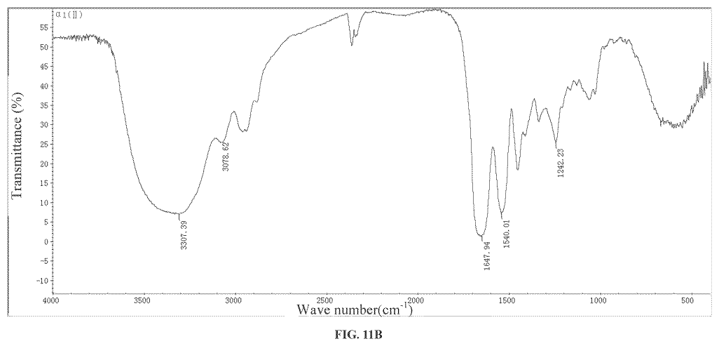

Infrared (IR) spectra of the purified a1 (I) M1, a1 (II) M6, a1 (I), and a1 (II) protein samples are shown in FIG. 11A-FIG. 11B and FIG. 12A-FIG. 12B. It can be seen that characteristic absorption average wave numbers of a1 (I) M1, a1 (II) M6, a1 (I), and a1 (II) all were in line with structural characteristics of recombinant collagen: an amide A (about 3,299 cm-1), an amide B (about 3,081 cm⁻¹), an amide I (about 1,650 cm⁻¹), an amide II (about 1,530 cm⁻¹ to 1,550 cm⁻¹), and an amide III (about 1,240 cm⁻¹), indicating that amino acid mutations in a1 (I) M1 and a1 (II) M6 did not affect the properties of collagen itself (as shown in literatures [1]. Chen Jingtao et al., Infrared Spectroscopy of Recombinant Collagen and Bovine Type I Collagen. Materials Reports, 2008 (03): p. 119-121; [2]. Doyle, B. B., E. G. Bendit and E. R. Blout, Infrared spectroscopy of collagen and collagen-like polypeptides. Biopolymers, 1975. 14 (5): p. 937-957; and [3]. Zhou Aimei et al., Isolation, Purification, and Structural Characterization of Recombinant Human Collagen. Food and Fermentation Industries, 2015 (03): p. 46-52.).

### (2) Detection of a Cell Adhesion Activity of Recombinant Collagens

A method for detecting a cell adhesion activity of recombinant collagen could be found in the literature: Juming Yao, Satoshi Yanagisawa, Tetsuo Asakura. Design, Expression and Characterization of Collagen-Like Proteins Based on the Cell Adhesive and Crosslinking Sequences Derived from Native Collagens, J Biochem. 136, 643-649 (2004). The detection of a cell adhesion activity was entrusted to the Functional Nanomaterials and Biomedical Testing Laboratory of School of Pharmacy, Changzhou University.

A specific implementation method was as follows: NIH/3T3 cells purchased from the Cell Bank of the Chinese Academy of Sciences (Product No. GNM6, the cultivation and passage methods were performed according to instructions of the cells) were cultivated normally. Lyophilized a1 (I) M1, a1 (II) M6. a1 (I), and a1 (II) recombinant collagen sponges, control human collagen (purchased from Sigma, Product No. C7774), and bovine serum albumin (BSA, purchased from Sangon Biotech (Shanghai) Co., Ltd.) each were taken and dissolved with ultrapure water or a 1 M HCl solution, then a protein concentration was determined according to a UV protein quantification empirical formula of C (mg/mL)=0.144×(A215-A225), and resulting solutions each were diluted with phosphate buffered saline (PBS) (pH 7.4) to 0.5 mg/mL. 100 μL of each of protein solutions and a blank PBS solution was added to a 96-well cell culture plate, and the plate was allowed to stand for 60 min at room temperature. Then NIH/3T3 cells in a well growth state were added at 10⁵ cells/well, and cultivated at 37° C. and 5% CO₂ for 60 min. Cells in each well were washed with PBS 4 times. An LDH detection kit (Roche, 04744926001) was used to detect the absorbance OD₄₉₂ nm (specific operations were performed with reference to instructions).

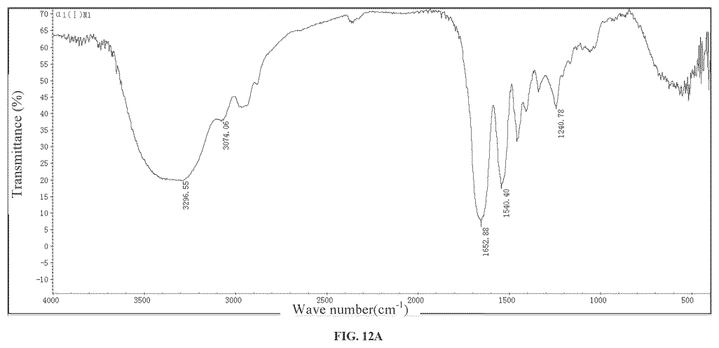

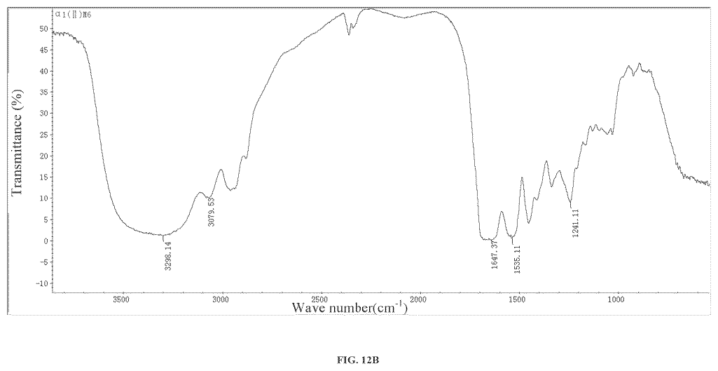

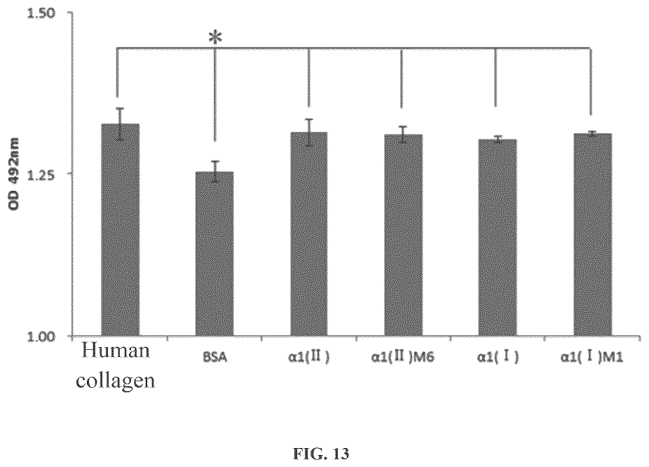

The absorbance OD₄₉₂ nm can correspondingly characterize a cell adhesion activity of a collagen sample. The higher the absorbance OD₄₉₂ nm, the more the cells to which the collagen adheres and the higher the cell adhesion activity, such that the collagen is more likely to help cells adhere to a wall or adhere to an extracellular matrix in a short time, which is conducive to building an excellent extracellular environment. As shown in FIG. 13, the recombinant collagen a1 (I) M1, a1 (II) M6, a1 (I), and a1 (II) all have a similar cell adhesion activity to commercially available native human collagen, and all have a significantly higher cell adhesion activity than the control group, and there is no significant difference between a1 (I) M1 and a1 (I) and between a1 (II) M6 and a1 (II) in terms of the cell adhesion activity.

### (3) Preparation and Detection of Recombinant Collagen Hydrogels

Recombinant collagens 1 (I) M1, a1 (II) M6, a1 (I), and a1 (II) each were taken and dissolved in water for injection at a concentration of 10% to obtain recombinant collagen solutions, a pH of the recombinant collagen solutions was controlled in a range of 4 to 6, and the recombinant collagen solutions were filtered through a 0.22 μm sterile filter for sterilization; 0.1 g of a sterile 10% (w/w)N-hydroxysuccinimide (NHS) solution was added per 1 g of a dry collagen powder to obtain first mixed solutions, and first mixed solutions each were thoroughly mixed; then 0.13 g of a sterile 50% (w/w) 1-ethyl-3-(-3-dimethylaminopropyl) carbodiimide hydrochloride (EDC) solution was added per 1 g of a dry collagen powder to obtain second mixed solutions; and the second mixed solutions each were allowed to stand at room temperature (20° C. to 30° C.) to allow a reaction for 2 h to 6 h to obtain hydrogels. The hydrogels each were subjected to dialysis in a sterile PBS solution (NaCl: 8.5 g/L, Na₂HPO₄: 0.5 g/L, and NaH₂PO₄: 0.15 g/L, pH: 7.2) continuously for 120 h, where a ratio of a hydrogel to a PBS dialysis solution was 1:6 (m/m), and a PBS dialysis solution was completely changed every 24 h to remove NHS and EDC residues. Purified hydrogels produced after the dialysis each were filled in a sterile container and placed at room temperature.

The purified hydrogels each were lyophilized to remove moisture, weighed, then placed in a sterile PBS solution for 24 h until each hydrogel completely absorbed water and swelled, and then taken out. The moisture on a surface of each swollen hydrogel was removed with absorbent paper, and then the swollen hydrogel was weighed. A swelling ratio was calculated with reference to the method in the literature: Q_{swelling ratio}=(W_{water-absorbed swollen gel weight}−W_{dry gel weight})/W_{dry gel weigh}t. The elasticity modulus (storage modulus, small amplitude frequency scan, 25° C., stress: 0.5%, and 0.1 rad/s to 100.0 rad/s) and a dynamic viscosity (flow peak hold, 25° C., and shear rate: 2.0 s′) of a hydrogel were measured by a rheometer (Discovery HR-2). A lyophilized hydrogel was transferred to liquid nitrogen, quickly frozen, broken, and subjected to surface scanning by SEM (Hitachi TM3030PLUS). Elasticity modulus, dynamic viscosity, and swelling ratio results were shown in Table 2. There is no significant difference between hydrogels prepared from a1 (I) M1 and a1 (I) and between hydrogels prepared from a1 (II) M6 and a1 (II) under the same conditions in terms of hydrodynamic properties.

TABLE 2
Detection results of elasticity moduli, dynamic viscosities,
and swelling ratios of four hydrogels

| Hydrogel | Elasticity modulus | Viscosity | Swelling ratio (g/g of a dry |
|---|---|---|---|
| type | (Pa) | (Pa · S) | hydrogel) |
| α1(II) | 104.63 | 82.87 | 14.18 |
| α1(II)M6 | 102.02 | 77.80 | 14.01 |
| α1(I) | 234.67 | 190.67 | 12.53 |
| α1(I)M1 | 292.68 | 170.97 | 12.22 |

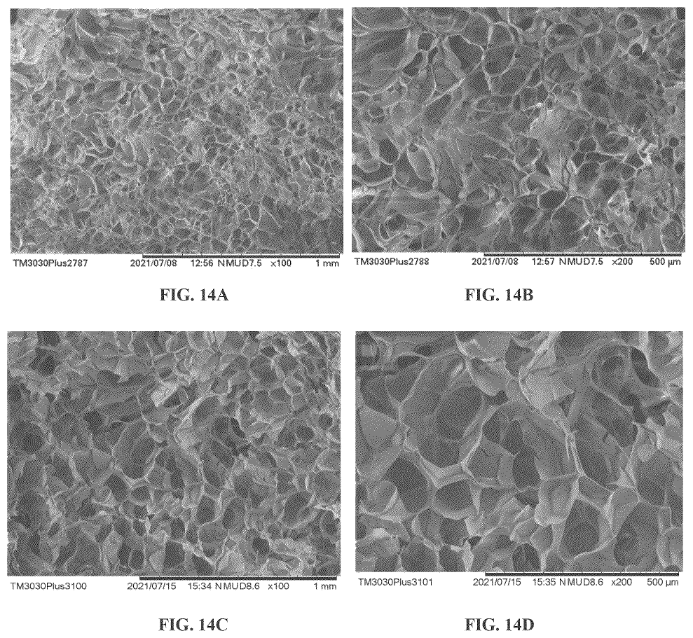

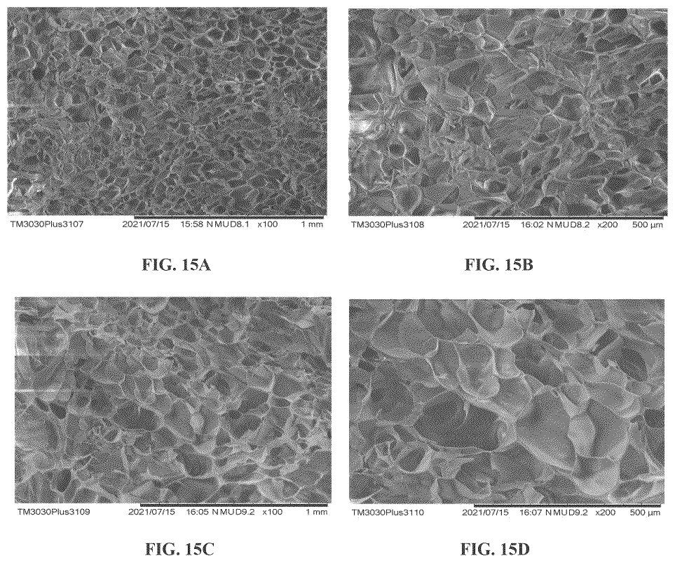

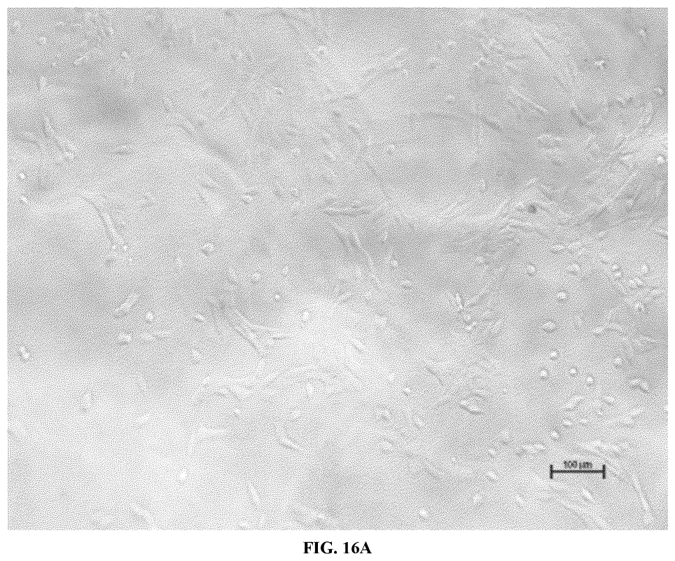

As shown in FIGS. 14A-14D and FIGS. 15A-15D, hydrogels prepared from a1 (I) M1 and a1 (II) M6 are the same as hydrogels prepared from a1 (I) and a1 (II), respectively, and all have a porous network structure with a pore size in a range of 100 μm to 200 μm, excellent permeability, and a spatial structure basis for retaining a large amount of moisture. Thus, the hydrogels prepared from a1 (I) M1 and a1 (II) M6 can provide a space for adhesion, support, growth, and migration of cells and serve as a channel for delivering nutrients and metabolites, and have the potential of being used in the field of biomedical materials.

### (4) Cell Detection of Recombinant Collagen Hydrogels

Hydrogels stored aseptically each were placed in a 24-well cell culture plate. NIH/3T3 cells cultivated normally purchased from the Cell Bank of the Chinese Academy of Sciences (Product No. GNM6, the cultivation and passage methods were performed according to instructions of the cells) were taken, washed with PBS, and digested with trypsin, then a medium was added to obtain a cell suspension, and the cell suspension was pipetted up and down for thorough mixing and then counted. The cell suspension was inoculated at 10⁵ cells/well into the cell culture plate coated with the hydrogels and cultivated for 24 h to 72 h, and the adhesion and proliferation of cells on the hydrogels were observed.

(1) A 24-well plate was taken, 1 mM Calcein AM (purchased from the Research Institute of Beyotime Biotechnology) solution was prepared with DMSO and diluted with D-PBS to obtain a 50 μM calcein working solution. The medium in wells of the cell culture plate was removed, and the wells were washed with PBS several times. Then 1 mL of a serum-containing DMEM medium and 100 μL of the Calcein AM solution ( 1/10 of the medium) were added to the cell culture plate, and the cell culture plate was incubated for 30 min to allow the staining of cells. Then the medium was changed, the cells were cultivated for 30 min, and the hydrogel was gently taken out, placed in a new culture well, and photographed under a fluorescence microscope (a maximum excitation wavelength was 494 nm and a maximum emission wavelength was 514 nm).

(2) Another 24-well plate was taken, 200 μL of an MTT solution (purchased from the Research Institute of Beyotime Biotechnology) was added to the 24-well plate, the NIH/3T3 cells were cultivated for 4 h, and the production of blue-purple crystals in the cells was observed. The medium was discarded, and the hydrogel was washed with PBS, cut longitudinally, placed in a new culture well, and photographed under a microscope.

The experiments in this example were entrusted to the Functional Nanomaterials and Biomedical Testing Laboratory of School of Pharmacy, Changzhou University.

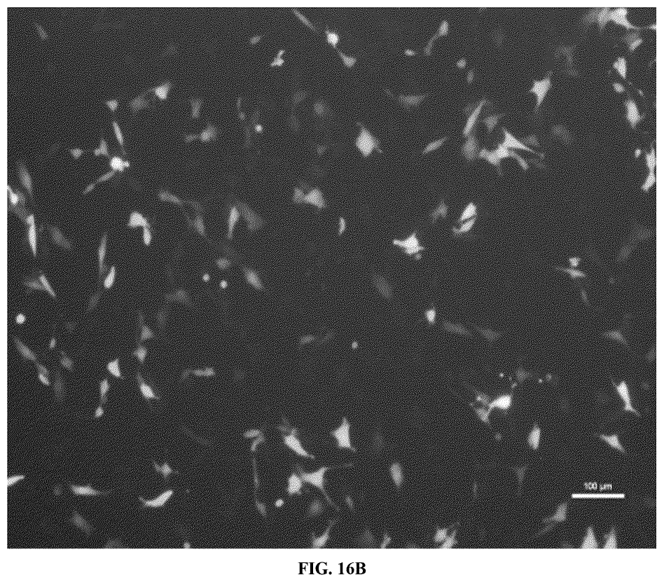

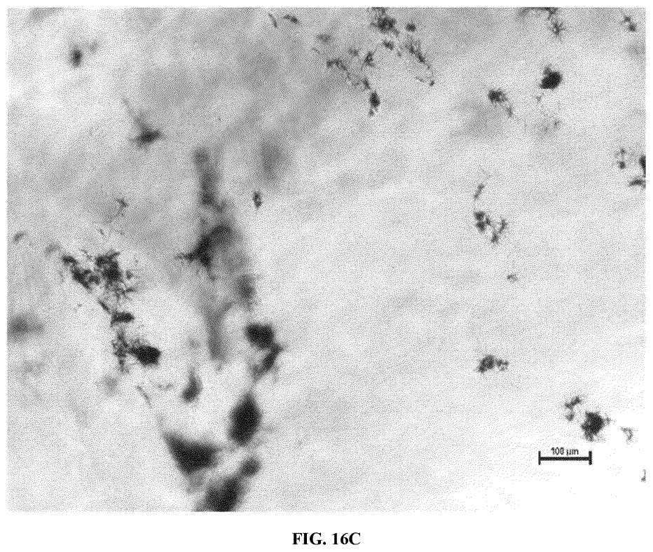

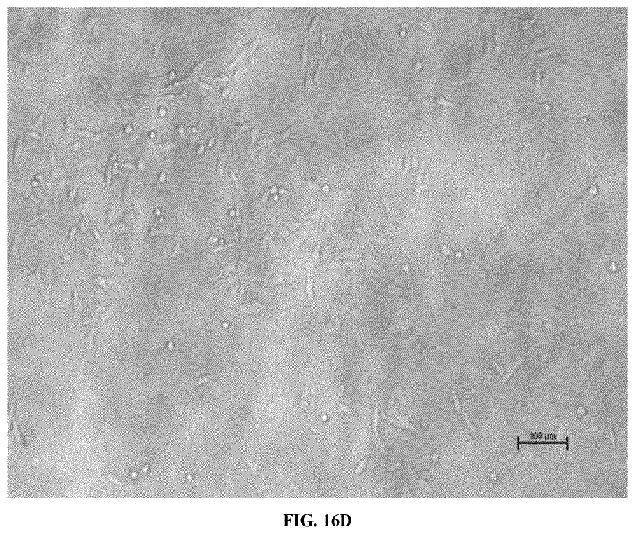

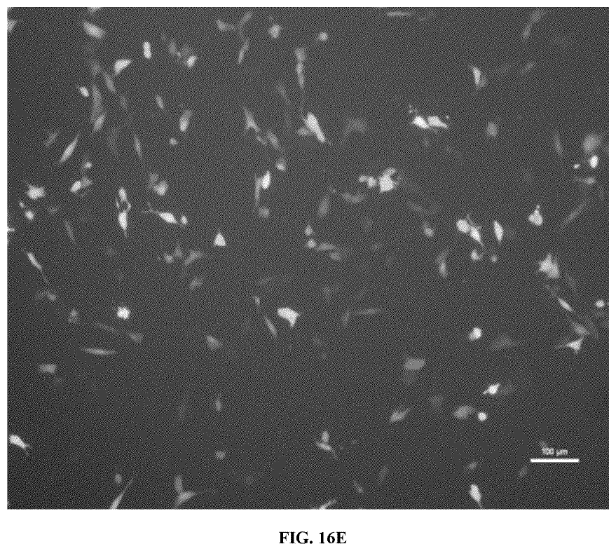

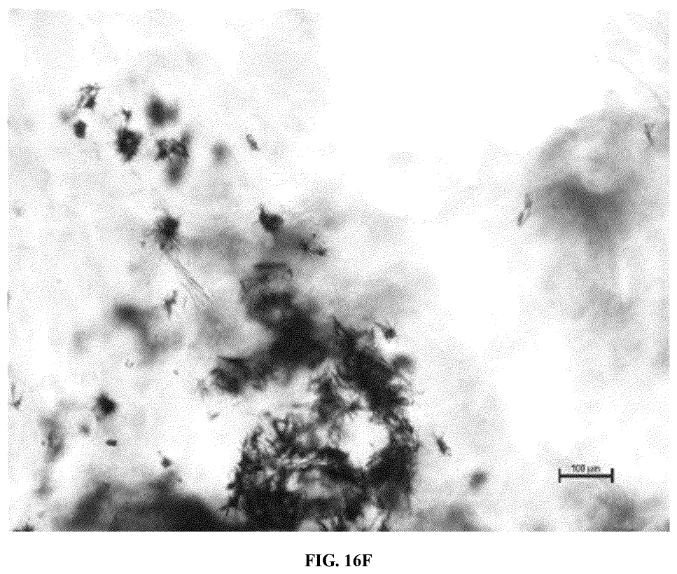

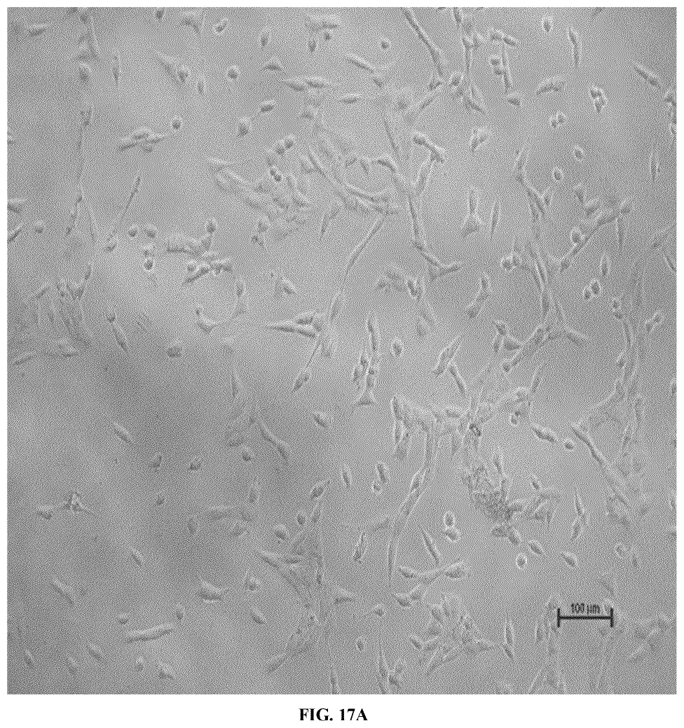

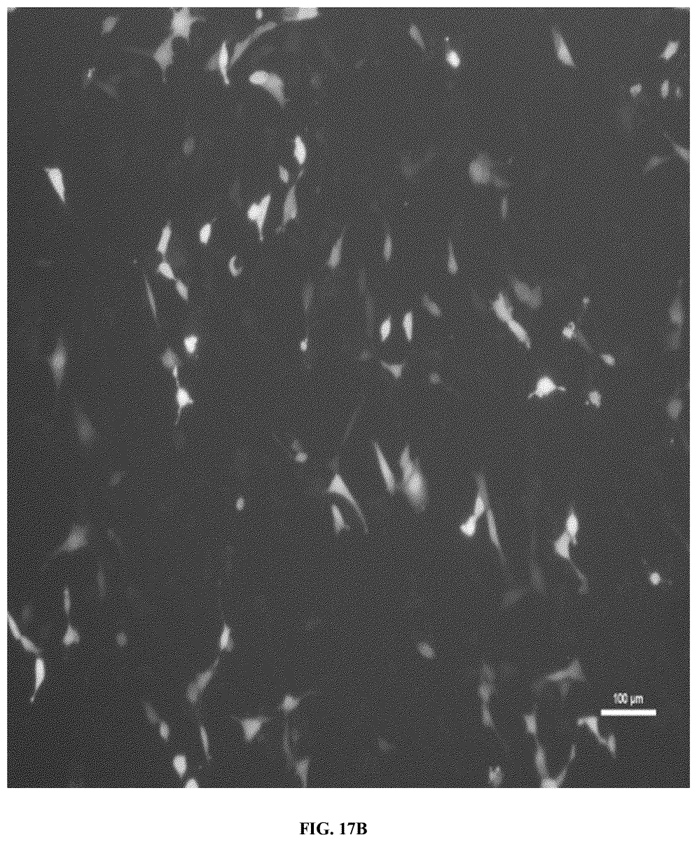

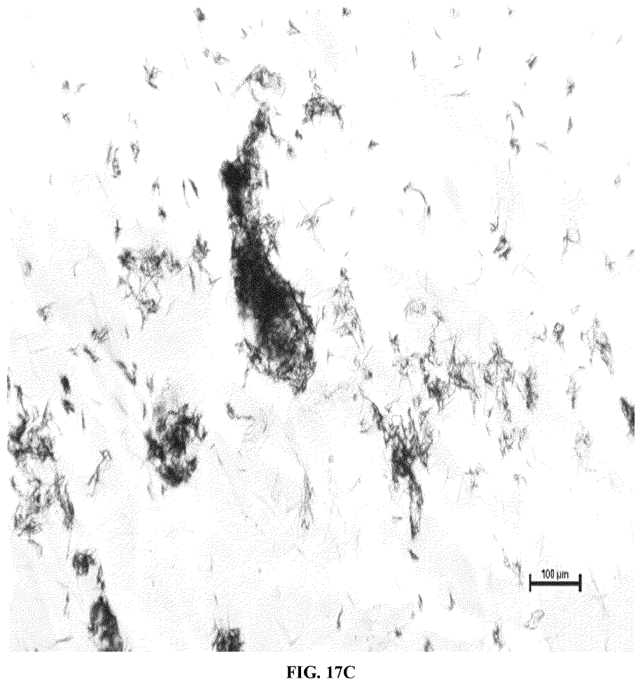

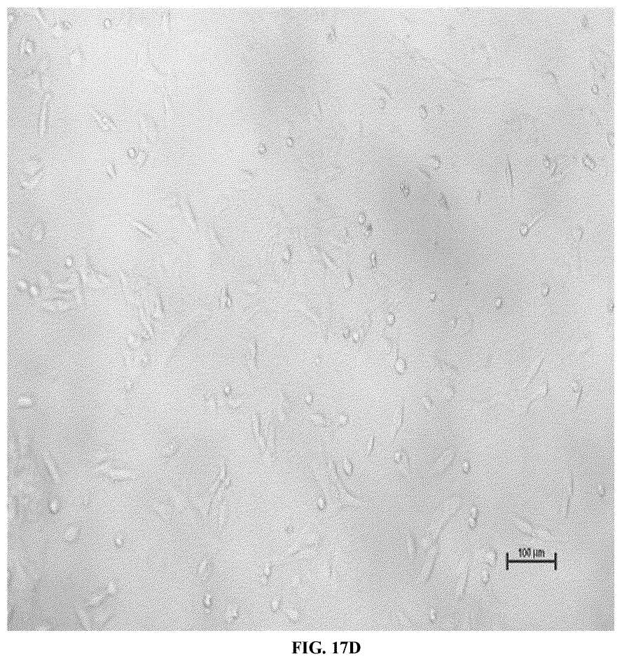

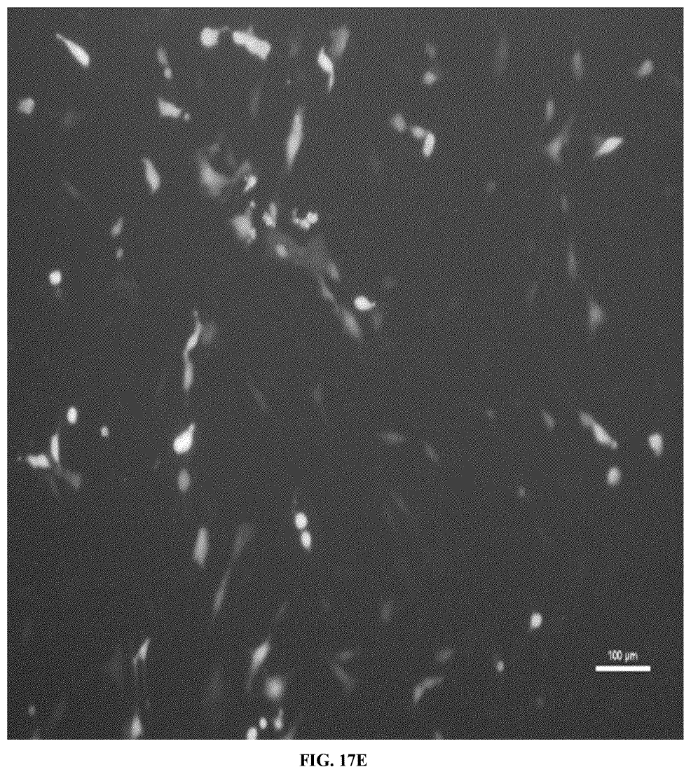

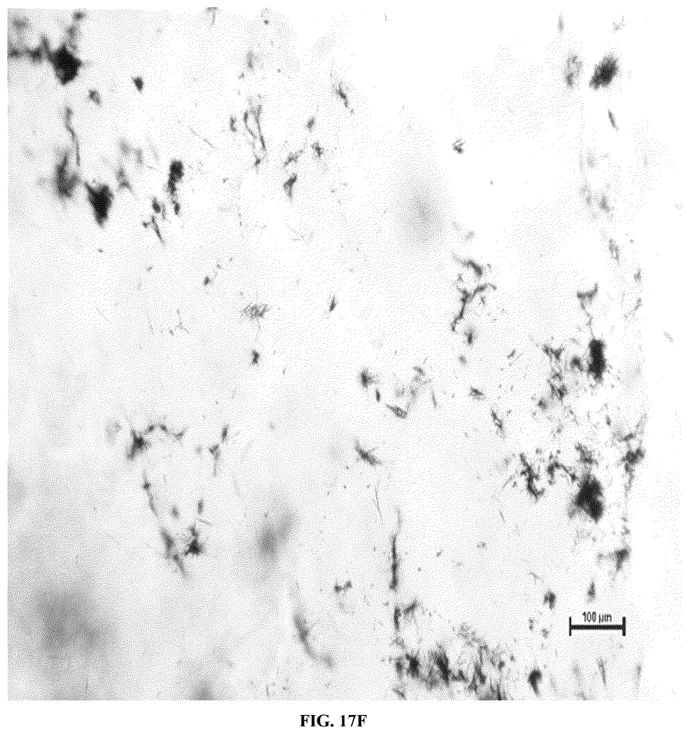

Results are shown in FIGS. 16A-16F and FIGS. 17A-17F. The hydrogels prepared from a1 (I) M1 and a1 (II) M6 are the same as the hydrogels prepared from a1 (I) and a1 (II), and NIH/3T3 cells have a normal morphology under a brightfield microscope, which is a typical fibroblast morphology. After NIH/3T3 cells growing adherently on a hydrogel are stained with Calcein AM, green fluorescence can be detected (bright parts in an image). After MTT is added to NIH/3T3 cells growing in a hydrogel, blue-purple crystals can be produced (dark parts in an image). The green fluorescence and the blue-purple crystals can only be produced by viable cells. It indicates that NIH/3T3 cells can adhere to, grow, and migrate normally in the hydrogels. The hydrogels prepared from a1 (I) M1 and a1 (II) M6 have similar biological functions to the hydrogels prepared from a1 (I) and a1 (II) with native sequences, and thus can be used as novel biomedical devices in fields such as wound healing and tissue regeneration.

What is claimed is:

1. A recombinant collagen a1 chain, wherein the recombinant collagen a1 chain is a1 (1) M1 or a1 (II) M6; the a1 (I) M1 is obtained through an amino acid mutation in a native full-length amino acid sequence of a human type I collagen a1 chain, and the a1 (II) M6 is obtained through an amino acid mutation in a native full-length amino acid sequence of a human type II collagen a1 chain; and the a1 (I) M1 has a sequence as set forth in SEQ ID NO: 2, and the a1 (II) M6 has a sequence as set forth in SEQ ID NO: 5.

2. A nucleotide encoding the recombinant collagen a1 chain according to claim 1.

3. The nucleotide according to claim 2, wherein the nucleotide sequence encoding the a1 (I) M1 is set forth in SEQ ID NO: 3; and the nucleotide sequence encoding the a1 (II) M6 is set forth in SEQ ID NO: 6.

4. A recombinant expression vector comprising the nucleotide according to claim 2.

5. An engineered strain expressing the recombinant collagen a1 chain according to claim 1, wherein the engineered strain is Pichia pastoris.

6. The engineered strain according to claim 5, wherein the engineered strain is deposited in the China General Microbiological Culture Collection Center (CGMCC), with an accession number of CGMCC NO. 21891 or CGMCC NO. 21892, wherein the engineered strain with the accession number of CGMCC NO. 21891 expresses the recombinant collagen a1 chain a1 (I) M1, and the engineered strain with the accession number of CGMCC NO. 21892 expresses the recombinant collagen a1 chain a1 (II) M6.

7. A use of the recombinant expression vector according to claim 4 in a preparation of the recombinant collagen a1 chain.

8. A preparation method of the recombinant collagen a1 chain according to claim 1, comprising the following steps:
(1) synthesizing a nucleotide sequence encoding the recombinant collagen a1 chain according to claim 1;
(2) constructing a recombinant expression vector;
(3) constructing engineered strains for an inducible expression, and screening the engineered strains to obtain an engineered strain with a high expression level;
(4) cultivating the engineered strain with the high expression level for a high-density fermentation to obtain a fermentation supernatant; and
(5) purifying the fermentation supernatant, followed by lyophilization, to obtain the recombinant collagen a1 chain.

9. The preparation method according to claim 8, wherein the engineered strain with the high expression level is Pichia pastoris, and is deposited in the China General Microbiological Culture Collection Center (CGMCC), with an accession number of CGMCC NO. 21891 or CGMCC NO. 21892.

10. A composition, comprising the recombinant collagen a1 chain according to claim 1.

11. An article, comprising the recombinant collagen a1 chain according to claim 1, or a composition comprising the recombinant collagen a1 chain, wherein the article is selected from the group consisting of a drug, a medical device, a biological material, a tissue-engineered product, and a cosmetic.

12. The article according to claim 11, wherein the article is a material for providing an adhesion, a support, and a growth and migration space for a cell or a material serving as a channel for delivering a nutrient and a metabolite.

13. The article according to claim 12, wherein the article is a collagen hydrogel.

14. A use of the recombinant collagen a1 chain according to claim 1, in a manufacture of a product, wherein the product is selected from the group consisting of a drug, a medical device, a biological material, a tissue-engineered product, and a cosmetic.

15. A use of the recombinant collagen a1 chain according to claim 1, in a manufacture of a product for promoting wound healing or tissue regeneration.

16. The use according to claim 14, wherein the product is a collagen hydrogel.

17. The use according to claim 15, wherein the product is a collagen hydrogel.

18. An engineered strain carrying the recombinant expression vector according to claim 4, wherein the engineered strain is Pichia pastoris.

19. The engineered strain according to claim 18, wherein the engineered strain is deposited in the China General Microbiological Culture Collection Center (CGMCC), with an accession number of CGMCC NO. 21891 or CGMCC NO. 21892, wherein the engineered strain with the accession number of CGMCC NO. 21891 expresses the recombinant collagen a1 chain a1 (I) M1, and the engineered strain with the accession number of CGMCC NO. 21892 expresses the recombinant collagen a1 chain a1 (II) M6.

20. A use of the engineered strain according to claim 5 in a preparation of the recombinant collagen a1 chain.

21. The use according to claim 20, wherein the engineered strain is deposited in the China General Microbiological Culture Collection Center (CGMCC), with an accession number of CGMCC NO. 21891 or CGMCC NO. 21892, wherein the engineered strain with the accession number of CGMCC NO. 21891 expresses the recombinant collagen a1 chain a1 (I) M1, and the engineered strain with the accession number of CGMCC NO. 21892 expresses the recombinant collagen a1 chain a1 (II) M6.

22. A use of the engineered strain according to claim 18 in a preparation of the recombinant collagen a1 chain.

23. The use according to claim 22, wherein the engineered strain is deposited in the China General Microbiological Culture Collection Center (CGMCC), with an accession number of CGMCC NO. 21891 or CGMCC NO. 21892, wherein the engineered strain with the accession number of CGMCC NO. 21891 expresses the recombinant collagen a1 chain a1 (I) M1, and the engineered strain with the accession number of CGMCC NO. 21892 expresses the recombinant collagen a1 chain a1 (II) M6.

24. A use of the nucleotide according to claim 2 in a manufacture of a product, wherein the product is selected from the group consisting of a drug, a medical device, a biological material, a tissue-engineered product, and a cosmetic.

25. The use according to claim 24, wherein the nucleotide sequence encoding the a1 (I) M1 is set forth in SEQ ID NO: 3; and the nucleotide sequence encoding the a1 (II) M6 is set forth in SEQ ID NO: 6.

26. A use of the recombinant expression vector according to claim 4 in a manufacture of a product, wherein the product is selected from the group consisting of a drug, a medical device, a biological material, a tissue-engineered product, and a cosmetic.

27. A use of the engineered strain according to claim 5 in a manufacture of a product, wherein the product is selected from the group consisting of a drug, a medical device, a biological material, a tissue-engineered product, and a cosmetic.

28. The use according to claim 27, wherein the engineered strain is deposited in the China General Microbiological Culture Collection Center (CGMCC), with an accession number of CGMCC NO. 21891 or CGMCC NO. 21892, wherein the engineered strain with the accession number of CGMCC NO. 21891 expresses the recombinant collagen a1 chain a1 (I) M1, and the engineered strain with the accession number of CGMCC NO. 21892 expresses the recombinant collagen a1 chain a1 (II) M6.

29. A use of the engineered strain according to claim 18 in a manufacture of a product, wherein the product is selected from the group consisting of a drug, a medical device, a biological material, a tissue-engineered product, and a cosmetic.

30. The use according to claim 29, wherein the engineered strain is deposited in the China General Microbiological Culture Collection Center (CGMCC), with an accession number of CGMCC NO. 21891 or CGMCC NO. 21892, wherein the engineered strain with the accession number of CGMCC NO. 21891 expresses the recombinant collagen a1 chain a1 (I) M1, and the engineered strain with the accession number of CGMCC NO. 21892 expresses the recombinant collagen a1 chain a1 (II) M6.

31. A use of the composition according to claim 10 in a manufacture of a product, wherein the product is selected from the group consisting of a drug, a medical device, a biological material, a tissue-engineered product, and a cosmetic.

32. A use of the nucleotide according to claim 2 in a manufacture of a product for promoting wound healing or tissue regeneration.

33. The use according to claim 32, wherein the nucleotide sequence encoding the a1 (I) M1 is set forth in SEQ ID NO: 3; and the nucleotide sequence encoding the a1 (II) M6 is set forth in SEQ ID NO: 6.

34. A use of the recombinant expression vector according to claim 4 in a manufacture of a product for promoting wound healing or tissue regeneration.

35. A use of the engineered strain according to claim 5 in a manufacture of a product for promoting wound healing or tissue regeneration.

36. The use according to claim 35, wherein the engineered strain is deposited in the China General Microbiological Culture Collection Center (CGMCC), with an accession number of CGMCC NO. 21891 or CGMCC NO. 21892, wherein the engineered strain with the accession number of CGMCC NO. 21891 expresses the recombinant collagen a1 chain a1 (I) M1, and the engineered strain with the accession number of CGMCC NO. 21892 expresses the recombinant collagen a1 chain a1 (II) M6.

37. A use of the engineered strain according to claim 18 in a manufacture of a product for promoting wound healing or tissue regeneration.

38. The use according to claim 37, wherein the engineered strain is deposited in the China General Microbiological Culture Collection Center (CGMCC), with an accession number of CGMCC NO. 21891 or CGMCC NO. 21892, wherein the engineered strain with the accession number of CGMCC NO. 21891 expresses the recombinant collagen a1 chain a1 (I) M1, and the engineered strain with the accession number of CGMCC NO. 21892 expresses the recombinant collagen a1 chain a1 (II) M6.

39. A use of the composition according to claim 10 in a manufacture of a product for promoting wound healing or tissue regeneration.
